SEQUENCE LISTING (12 sequences)

SEQ ID NO: 1 - protein, 14 aa, Homo sapiens
  MISC_FEATURE (4)..(4): deamidation of N due to PNGaseF treatment
AGPNGTLFVADAYK

SEQ ID NO: 2 - protein, 17 aa, Homo sapiens
  MISC_FEATURE (5)..(5): deamidation of N due to PNGaseF treatment
NLEKNSTKQEILAALEK

SEQ ID NO: 3 - protein, 20 aa, Homo sapiens
  MISC_FEATURE (1)..(1): iTRAQ modification
VPDSCCINVTVGCGINFNEK

SEQ ID NO: 4 - protein, 18 aa, Homo sapiens
  MISC_FEATURE (1)..(1): iTRAQ modification
QQMENYPKNNHTASILDR

SEQ ID NO: 5 - protein, 10 aa, Homo sapiens
  MISC_FEATURE (5)..(5): deamidation of N due to PNGaseF treatment
VQPFNVTQGK

SEQ ID NO: 6 - protein, 17 aa, Homo sapiens
  MISC_FEATURE (12)..(12): deamidation of N due to PNGaseF treatment
VPYNVGPGFTGNFSTQK

SEQ ID NO: 7 - protein, 11 aa, Homo sapiens
  misc_feature (3)..(3): deamidation of N due to PNGaseF treatment
YYNYTLSINGK

SEQ ID NO: 8 - protein, 23 aa, Homo sapiens
  misc_feature (3)..(3): deamidation of N due to PNGaseF treatment
HNNDTQHIWESDSNEFSVIADPR

SEQ ID NO: 9 - protein, 19 aa, Homo sapiens
  misc_feature (2)..(2): deamidation of N due to PNGaseF treatment
TNSTFVQALVEHVKEECDR

SEQ ID NO: 10 - protein, 14 aa, Homo sapiens
  misc_feature (2)..(2): deamidation of N due to PNGaseF treatment
TNSTFVQALVEHVK

SEQ ID NO: 11 - protein, 9 aa, Homo sapiens
  misc_feature (5)..(5): deamidation of N due to PNGaseF treatment
LIDNNKTEK

SEQ ID NO: 12 - protein, 25 aa, Homo sapiens
  misc_feature (13)..(13): deamidation of N due to PNGaseF treatment
DVVTAAGDMLKDNATEEEILVYLEK

(19) United States
(12) Patent Application Publication
(10) Pub. No.: US 2016/0069884 A1
(43) Pub. Date: Mar. 10, 2016

(54) BIOMARKERS FOR DISTINGUISHING BETWEEN AGGRESSIVE PROSTATE CANCER AND NON-AGGRESSIVE PROSTATE CANCER

(71) Applicant: THE JOHNS HOPKINS UNIVERSITY, Baltimore, MD (US)

(72) Inventors: Hui Zhang, Ellicott City, MD (US); Punit Shah, Baltimore, MD (US); Qing Kay Li, Baltimore, MD (US); Daniel W. Chan, Clarksville, MD (US)

(21) Appl. No.: 14/848,830

(22) Filed: Sep. 9, 2015

Related U.S. Application Data
(60) Provisional application No. 62/047,678, filed on Sep. 9, 2014.

(51) Int. Cl. G01N 33/574

(52) U.S. Cl. CPC G01N 33/57434

(57) ABSTRACT

The present invention relates to the field of biomarkers. More specifically, the present invention relates to biomarkers useful in diagnosing aggressive prostate cancer. In one embodiment, a method for identifying patients as having or likely to have aggressive prostate cancer comprises the steps of (a) performing an assay on a biological sample obtained from the patient to detect fucosylated prostate specific antigen (PSA), transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B; and (b) identifying the patient as having or likely to have aggressive prostate cancer if there is a statistically significant difference in the levels of fucosylated PSA, transmembrane prostate androgen-induced protein, kallikrein-2, lipid phosphate phosphohydrolase 3, heparan-sulfate 6-O-sulfotransferase, PAP, nuclear RNA export factor 2, and protein POF1B as compared to corresponding levels in a control sample that correlates to non-aggressive prostate cancer.

### CROSS-REFERENCE TO RELATED APPLICATIONS

This application claims the benefit of U.S. Provisional Application No. 62/047,678, filed Sep. 9, 2014, which is incorporated herein by reference in its entirety.

### STATEMENT OF GOVERNMENTAL INTEREST

This invention was made with government support under grant no. U24CA160036, grant no. U01CA152813, grant no. U24CA115102, and grant no. P01HL107153, awarded by the National Institutes of Health. The government has certain rights in the invention.

### FIELD OF THE INVENTION

The present invention relates to the field of biomarkers. More specifically, the present invention relates to biomarkers useful in diagnosing aggressive prostate cancer.

### INCORPORATION-BY-REFERENCE OF MATERIAL SUBMITTED ELECTRONICALLY

This application contains a sequence listing. It has been submitted electronically via EFS-Web as an ASCII text file entitled “P13234-02_ST25.txt.” The sequence listing is 4,497 bytes in size, and was created on Sep. 9, 2015. It is hereby incorporated by reference in its entirety.

### BACKGROUND OF THE INVENTION

Prostate cancer (Pca) is the most common cancer of men in the United States and worldwide. Although the estimated new cases in the U.S. will exceed 200,000 annually, the majority of Pca is presented as a localized and/or slow-growing disease, which does not need invasive treatments. Currently, prostate-specific antigen (PSA) is the most used serum biomarker for the detection of Pca in high risk populations. However, there is a debate regarding its clinical usefulness and benefit for prostate cancer patients. The European Randomized Study of Screening for Prostate Cancer (ERSPC) revealed a 20% reduction of mortality in prostate cancer patients, but also demonstrated a high overdiagnostic rate in the screening populations. In contrast, the United States Prostate, Lung, Colorectal, and Ovarian (PLCO) cancer screening trail revealed no difference of cumulative mortality rates between screening population and controls. Recently, the U.S. Preventive Services Task Force (USPSTF) has recommended against serum PSA-based screening for Pca (D recommendation). Limitations of serum PSA, such as lack of sensitivity and specificity, are well known during the clinical practice. It is also well known that the outcome of Pca patients is correlated with the clinical behavior of the tumor, and the serum PSA cannot be used reliably to separate slow-growing tumors from aggressive, fatal tumors in Pca patients. Thus, it causes the clinical problem of under-treatment of aggressive tumors (AG), and the over-treatment of non-aggressive tumors (NAG).

In recent years, tremendous efforts have been focused on the discovery of novel biomarkers to improve the detection of Pca, particularly the detection of aggressive subtypes of Pca from slow growing non-aggressive subtypes. Although several new biomarkers have emerged and/or been reported including serum markers of kallikrein 2, early prostate cancer antigen (EPCA), prostate cancer gene 3 (PCA3), tissue markers of urokinase-type plasminogen activator receptor (uPAR), α-methylacyl-CoA-racemase (AMACR), urine markers of uPAR, PCA3, TMPRSS2-ERG, and others, clinical utilities of the majority of these markers are still under evaluation or in the validation phase. Other clinical tests including both non-invasive tests such as proPSA as part of the prostate health index (phi, approved by FDA (U.S. Food and Drug Administration)), and invasive test (using tumor tissue) such as oncotype dx and prolaris score (offered by CLIA certified lab), have been used in the clinical practice. However, none of these markers and/or tests including serum PSA can be reliably used to distinguish AG from NAG Pca.

### SUMMARY OF THE INVENTION

The present invention is based, at least in part, on the identification of proteins whose expression is significantly altered in aggressive prostate tumors. In one aspect, the present invention provides assays useful for identifying patients as having or likely to have aggressive prostate cancer. The assays can be singleplex or multiplex assays. In certain embodiments, the assay utilizes antibodies to capture biomarker proteins of interest. The assays can further use antibodies to detect and quantify biomarker proteins of interest. The assay can also use lectins to enrich, isolate or otherwise select for the biomarker proteins of interest followed by subsequent analysis using antibodies. In certain embodiments, the assay utilizes both antibodies and lectins to detect and measure biomarker proteins of interest. In certain embodiments, the assay utilizes lectins or antibodies to enrich target proteins and followed by mass spectrometric analysis. In certain embodiments, the assay utilizes mass spectrometry to quantify the proteins or modified glycoproteins with fucoylated glycans.

The methods described herein can be used to measure the biomarker proteins and modified forms thereof including glycosylated forms (e.g., fucosylated). For example, an assay can measure total PSA, free PSA and fucosylated PSA (and optionally a percentage of fucosylated PSA can be determined). In a broader sense, the assays herein can be used or adjusted to measure total levels of a given protein and levels of a modified version of the protein (PSA and fucosylated PSA). Total glycoprotein levels, specific levels of glycoproteins and the like can be measured.

In one embodiment, a method comprises (a) isolating glycoproteins from a serum sample obtained from the patient using a lectin affinity capture assay; and (b) quantitating the amount of fucosylated PSA from the isolated glycoproteins of step (a) using an immunoassay. In another embodiment, the method further comprises the step of generating a report showing the quantitated amount of fucosylated PSA. In yet another embodiment, the method further comprises a display of control levels of fucosylated PSA that correlate to aggressive prostate cancer and non-aggressive prostate cancer. In alternative embodiments, the method can further comprise the step of identifying the patient as having aggressive prostate cancer based on a statistically significant increase in fucosylated PSA present in the patient sample relative to reference levels that correlate to non-aggressive prostate cancer. In certain embodiments, the method further comprises the step of recommending, prescribing or treating the patient with, an appropriate therapeutic regimen for aggressive prostate cancer if the quantitated amount of fucosylated PSA correlates to aggressive prostate cancer or recommending, prescribing or treating the patient with, an appropriate therapeutic regimen for non-aggressive prostate cancer if the quantitated amount of fucosylated PSA correlates to non-aggressive prostate cancer. It is also contemplated that the methods comprise a recommendation, prescription, treatment or administration of a cancer therapy to a patient identified or diagnosed as having aggressive prostate cancer or non-aggressive prostate cancer using a method described herein.

In one aspect, the present invention provides multiplex assays for distinguishing aggressive from non-aggressive prostate cancer in a patient. In particular embodiments, the method comprises the steps of (a) incubating a sample comprising biomarker glycoproteins of interest obtained from a patient with a plurality of lectins that specifically bind glycoproteins; (b) adding a plurality of monoclonal antibodies that specifically bind the biomarker glycoproteins of interest; (c) detecting the lectin-bound biomarker glycoproteins using a labeled detection antibody that binds the lectin-bound biomarker glycoproteins; and (d) identifying the patient as having aggressive prostate cancer if the detected biomarker glycoproteins are statistically significantly changed relative to reference levels that correlate to non-aggressive prostate cancer. In a specific embodiment, the biomarker glycoproteins of interest comprise fucosylated prostate specific antigen (PSA).

In another embodiment, the biomarker glycoproteins of interest comprise one or more of PSA, UDP-glucuronosyltransferase, 2′-5′-oligoadenylate synthase-like protein, alpha-(1,6)-fucosyltransferase, annexin A1, G-protein coupled receptor 126, anterior gradient protein 2 homolog, calcium/calmodulin-dependent protein kinase II inhibitor 1, desmocollin-2 isoform Dsc2a preproprotein, interferon-induced protein with tetratricopeptide repeats 2, matrix metalloproteinase-15 preproprotein, interferon-induced protein with tetratricopeptide repeats 1, thymidylate synthase, interferon-induced protein with tetratricopeptide repeats 3, histone chaperone ASF1B, cyclin-dependent kinases regulatory subunit 2, nucleolar and spindle-associated protein 1, membrane associated phospholipase A2, ubiquitin-conjugating enzyme E2 C, solute carrier family 22 member 3, G2/mitotic-specific cyclin-B1, paternally-expressed gene 3 protein, PCNA-associated factor, paternally-expressed gene 3 protein, redox-regulatory protein FAM213A, kinesin family member 20A, cytosolic thymidine kinase, protein FAM83D, importin subunit alpha-1, transferrin receptor protein 1, hyaluronan mediated motility receptor, lymphokine-activated killer T-cell-originated protein kinase, transmembrane prostate androgen-induced protein, kallikrein-2 isoform 2 preproprotein, lipid phosphate phosphohydrolase 3, heparan-sulfate 6-O-sulfotransferase 1, prostatic acid phosphatase isoform PAP, nuclear RNA export factor 2, protein POF1B, and glycosylated forms thereof (including, for example, fucosylated PSA).

In particular embodiments, the patient sample is a serum, plasma, urine or tissue sample. In certain embodiments, the biomarker glycoproteins of interest comprise fucosylated PSA, transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B.

In a specific embodiment, a multiplex assay for distinguishing aggressive from non-aggressive prostate cancer in a patient comprises the steps of (a) incubating a sample comprising biomarker glycoproteins of interest obtained from a patient with a plurality of lectins that specifically bind glycoproteins, wherein the biomarker glycoproteins of interest comprise one or more of PSA, UDP-glucuronosyltransferase, 2′-5′-oligoadenylate synthase-like protein, alpha-(1,6)-fucosyltransferase, annexin A1, G-protein coupled receptor 126, anterior gradient protein 2 homolog, calcium/calmodulin-dependent protein kinase II inhibitor 1, desmocollin-2 isoform Dsc2a preproprotein, interferon-induced protein with tetratricopeptide repeats 2, matrix metalloproteinase-15 preproprotein, interferon-induced protein with tetratricopeptide repeats 1, thymidylate synthase, interferon-induced protein with tetratricopeptide repeats 3, histone chaperone ASF1B, cyclin-dependent kinases regulatory subunit 2, nucleolar and spindle-associated protein 1, membrane associated phospholipase A2, ubiquitin-conjugating enzyme E2 C, solute carrier family 22 member 3, G2/mitotic-specific cyclin-B1, paternally-expressed gene 3 protein, PCNA-associated factor, paternally-expressed gene 3 protein, redox-regulatory protein FAM213A, kinesin family member 20A, cytosolic thymidine kinase, protein FAM83D, importin subunit alpha-1, transferrin receptor protein 1, hyaluronan mediated motility receptor, lymphokine-activated killer T-cell-originated protein kinase, transmembrane prostate androgen-induced protein, kallikrein-2 isoform 2 preproprotein, lipid phosphate phosphohydrolase 3, heparan-sulfate 6-O-sulfotransferase 1, prostatic acid phosphatase isoform PAP, nuclear RNA export factor 2, protein POF1B, and glycosylated forms thereof; (b) adding a plurality of monoclonal antibodies that specifically bind the biomarker glycoproteins of interest; (c) detecting the lectin-bound biomarker glycoproteins using a labeled detection antibody that binds the lectin-bound biomarker glycoproteins; and (d) identifying the patient as having aggressive prostate cancer if the detected biomarker glycoproteins are statistically significantly changed relative to reference levels that correlate to non-aggressive prostate cancer.

In another embodiment, a method for identifying patients as having or likely to have aggressive prostate cancer comprises the steps of (a) obtaining a biological sample from the patient; (b) performing an assay on the biological sample to detect fucosylated prostate specific antigen (PSA), transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B; and (c) identifying the patient as having or likely to have aggressive prostate cancer if there is a statistically significant difference in the levels of fucosylated PSA, transmembrane prostate androgen-induced protein, kallikrein-2, lipid phosphate phosphohydrolase 3, heparan-sulfate 6-O-sulfotransferase, PAP, nuclear RNA export factor 2, and protein POF1B as compared to corresponding levels in a control sample that correlates to non-aggressive prostate cancer. In other embodiments, the assay step detects fucosylated prostate specific antigen (PSA) and one or more of transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B.

In other embodiments, a multiplex assay for distinguishing aggressive from non-aggressive prostate cancer in a patient comprises the steps of (a) incubating a sample comprising biomarker proteins of interest obtained from a patient with a plurality of binding agents that specifically bind the biomarker proteins of interest; (b) detecting the biomarker proteins using an immunoassay or mass spectrometry; and (d) identifying the patient as having aggressive prostate cancer if there is a statistically significant difference in the levels of the detected biomarker proteins as compared to corresponding levels in a control sample that correlates to non-aggressive prostate cancer. In particular embodiments, the biomarker proteins of interest comprise unmodified forms and/or post-translationally modified forms. Post-translationally modifications can include fucosylation, glycosylation, citrullination, oxidation, methylation, phosphorylation, cysteinylation s-nitrosation, s-glutathyolation, or a combination thereof.

In one embodiment, a method for identifying patients as having or likely to have aggressive prostate cancer comprises the steps of (a) obtaining a biological sample from the patient; (b) performing an assay on the biological sample to detect fucosylated prostate specific antigen (PSA); and (c) identifying the patient as having or likely to have aggressive prostate cancer if there is a statistically significant difference in the levels of fucosylated PSA as compared to corresponding levels in a control sample that correlates to non-aggressive prostate cancer. In another embodiment, a method for identifying aggressive prostate cancer in a patient comprises the steps of (a) measuring the levels of one or more biomarkers in a sample collected from the patient; and (b) comparing the levels of the one or more biomarkers with predefined levels of the same biomarkers that correlate to a patient having aggressive prostate cancer and predefined levels of the same biomarkers that correlate to a patient not having aggressive prostate cancer, wherein a correlation to one of the predefined levels provides the identification.

In specific embodiments, the one or more biomarkers comprises fucosylated PSA. In other embodiments, the one or more biomarkers comprises fucosylated PSA and one or more of sTIE-2, sVEGFR-1, fucosylated TIMP-1, fucosylated DPP-4, and FUT8. The sample can be a tissue, urine, blood, plasma, serum sample, or other body fluids. In certain embodiments, the sample is a serum sample. In particular embodiments, the measuring step is performed using an immunoassay. In other embodiments, the measuring step is performed using mass spectrometry. In a specific embodiment, the correlation to a patient not having aggressive prostate cancer refers to a patient having non-aggressive prostate cancer. In another specific embodiment, the correlation to a patient not having aggressive prostate cancer refers to a patient not having cancer.

In another embodiment, the present invention provides a method for diagnosing aggressive prostate cancer in a patient comprising the steps of (a) collecting a serum sample from the patient; (b) detecting the levels of a panel of biomarkers in the sample collected from the patient, wherein the panel of biomarkers comprises fucosylated PSA; and (c) comparing the levels of the panel of biomarkers with predefined levels of the same panel of biomarkers that correlate to a patient having aggressive prostate cancer and predefined levels of the same panel of biomarkers that correlate to a patient not having aggressive prostate cancer, wherein a correlation to one of the predefined levels provides the diagnosis.

In another aspect, the present invention provides methods for treating prostate cancer in a patient. In a specific embodiment, the method comprises the steps of (a) collecting a serum sample from the patient; (b) detecting the levels of a panel of biomarkers in the sample collected from the patient, wherein the panel of biomarkers comprises fucosylated PSA; (c) comparing the levels of the panel of biomarkers with predefined levels of the same panel of biomarkers that correlate to a patient having aggressive prostate cancer and predefined levels of the same panel of biomarkers that correlate to a patient not having aggressive prostate cancer, wherein a correlation to one of the predefined levels provides the diagnosis; and (d) treating the patient with an appropriate therapeutic regimen for aggressive prostate cancer if the diagnosis of the patient correlates to aggressive prostate cancer or treating the patient with an appropriate therapeutic regimen for non-aggressive prostate cancer if the diagnosis of the patient correlates to non-aggressive prostate cancer. The appropriate therapeutic regimen (for aggressive prostate cancer or for non-aggressive prostate cancer) can be determined by one of ordinary skill in the art using the methods described herein and other patient and diagnostic information.

In yet another aspect, the present invention provides methods for determining the aggressive prostate cancer status in a patient. In a specific embodiment, the method comprises the steps of (a) collecting a sample from the patient; (b) measuring the levels of a panel of biomarkers in the sample collected from the patient, wherein the panel of biomarkers comprises fucosylated PSA; and (c) comparing the levels of the panel of biomarkers with predefined levels of the same panel of biomarkers that correlate to one or more aggressive prostate cancer statuses selected from the group consisting of having aggressive prostate cancer, not having aggressive prostate cancer, progressing aggressive prostate cancer, and regressing aggressive prostate cancer, wherein a correlation to one of the predefined levels determines the aggressive prostate cancer status of the patient.

In another embodiment, the present invention provides a multiplex assay for distinguishing aggressive from non-aggressive prostate cancer comprising the steps of (a) incubating a sample comprising biomarker proteins of interest obtained from a patient with a mixture of magnetic beads coupled with monoclonal antibodies that specifically bind the biomarker proteins of interest, wherein the biomarker proteins of interest comprise fucosylated PSA; (b) adding a mixture of biotinylated lectins that specifically bind fucosylated PSA; (c) adding a streptavidin labeled fluorescent marker that binds the biotinylated lectins bound to fucosylated PSA; (d) detecting fucosylated PSA; and (e) identifying the patient as having aggressive prostate cancer if the fluorescence intensity of fucosylated PSA is statistically significantly increased relative to a reference that correlates to non-aggressive prostate cancer. In specific embodiments, the fluorescent marker is phycoerythrin. In other embodiments, the sample comprises about 300 ng of protein. In a specific embodiment, the sample comprises less than about 400 ng of protein. The sample can comprises about 100 ng to about 1 mg of protein.

In particular embodiments, the biomarker proteins further comprise fucosylated tissue inhibitor of metallopeptidase 1 (TIMP-1), fucosylated dipeptidyl peptidase-IV (DPP-4), soluble form of the TIE-2 receptor (sTIE-2), soluble form of the vascular endothelial growth factor receptor 1 (sVEGFR-1), and alpha (1,6) fucosyltransferase (FUT8). In such embodiments, the multiplex assay can further comprise adding a mixture of biotinylated detection antibodies; adding a streptavidin labeled fluorescent marker that binds the biotinylated antibodies bound to TIMP-1, DPP-4, sTIE-2, sVEGFR-1, and FUT8; detecting TIMP-1, DPP-4, sTIE-2, sVEGFR-1, and FUT8; and identifying the patient as having aggressive prostate cancer if the fluorescence intensity of TIMP-1, DPP-4, sTIE-2, FUT8, fucosylated TIMP-1 and fucosylated DPP-4 is statistically significantly increased relative to a reference and the fluorescence intensity of sVEGFR-1 is statistically significantly decreased relative to a reference. The biotinylated detection antibodies can be immunoglobulin G antibodies.

In further embodiments, the biomarker proteins further comprise one or more of UDP-glucuronosyltransferase, 2′-5′-oligoadenylate synthase-like protein, alpha-(1,6)-fucosyltransferase, annexin A1, G-protein coupled receptor 126, anterior gradient protein 2 homolog, calcium/calmodulin-dependent protein kinase II inhibitor 1, desmocollin-2 isoform Dsc2a preproprotein, interferon-induced protein with tetratricopeptide repeats 2, matrix metalloproteinase-15 preproprotein, interferon-induced protein with tetratricopeptide repeats 1, thymidylate synthase, interferon-induced protein with tetratricopeptide repeats 3, histone chaperone ASF1B, cyclin-dependent kinases regulatory subunit 2, nucleolar and spindle-associated protein 1, membrane associated phospholipase A2, ubiquitin-conjugating enzyme E2 C, solute carrier family 22 member 3, G2/mitotic-specific cyclin-B1, paternally-expressed gene 3 protein, PCNA-associated factor, paternally-expressed gene 3 protein, redox-regulatory protein FAM213A, kinesin family member 20A, cytosolic thymidine kinase, protein FAM83D, importin subunit alpha-1, transferrin receptor protein 1, hyaluronan mediated motility receptor, lymphokine-activated killer T-cell-originated protein kinase, transmembrane prostate androgen-induced protein, kallikrein-2 isoform 2 preproprotein, lipid phosphate phosphohydrolase 3, heparan-sulfate 6-O-sulfotransferase 1, prostatic acid phosphatase isoform PAP, nuclear RNA export factor 2, protein POF1B.

In another aspect the present invention provides kit. In particular embodiments, a kit comprises (a) monoclonal antibodies that each specifically bind a biomarker protein of interest; (b) biotinylated immunoglobulin G detection antibodies; (c) biotinylated lectins that specifically bind glycosylated forms of the biomarker protein of interest; and (d) streptavidin labeled fluorescent markers. In a further embodiment, the kit further comprises (e) magnetic beads for conjugating to monoclonal antibodies that each specifically bind a biomarker protein of interest. In a specific embodiment, the biomarker protein of interest comprises PSA and the glycosylated form of PSA comprises fucosylated PSA. In another embodiment, the biomarker protein of interest further comprises one or more of transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B. In a more specific embodiment, the glycosylated form of the one or more biomarker proteins of interest is fucosylated.

### BRIEF DESCRIPTION OF THE FIGURES

FIG. 1A-FIG. 1B. The detection of glycoproteins and their fucosylated forms in sera from Pca patients. A, workflow of multiplex immunoassay. B, standard curves of immunoassays for the candidate glycoproteins.

FIG. 2A-FIG. 2I. The correlation of serum levels of PSA, TIMP1 and tPA with tumor Gleason scores. Line indicates mean value.

FIG. 3A-FIG. 3F. The correlation of serum levels of PSA and TIMP1 with tumor Gleason scores. Line indicates mean value.

FIG. 4. The correlation coefficient of total serum PSA versus fucosylated with different Gleason scores.

FIG. 5. Serum PSA and TIMP1 in the separation of Gleason score 6 and Gleason score 7-9 tumors by receive operating characteristic (ROC) analysis.

FIG. 6. Serum PSA and TIMP1 in the separation of Gleason score 6 and Gleason score 8-9 by receive operating characteristic (ROC) analysis.

FIG. 7. Schematic representation of the workflow for the integrated analysis of glycosite-containing peptides, global protein expression, and intact glycopeptides. Proteins were obtained from LNCap and PC3 cell lines followed by tryptic digestion and iTRAQ labeling. Labeled peptide samples were then combined and separated into two aliquots. One part is enriched for glycosite-containing peptides using Solid Phase Extraction of Glycopeptides (SPEG) and the other part was used for bRPLC separation and analysis of global proteomics and glycopeptides. Finally peptides were analyzed using LC-MS/MS.

FIG. 8. Distribution of proteins and glycoproteins between a replicate analyses of LNCaP (top panel) and the distribution of glycoproteins over proteins between LNCaP and PC3 indicating changes in glycosylation occupancy.

FIG. 9. Identification and quantification of CD63 total proteins and glycoproteins. A) Representative MS/MS spectrum of CD63 tryptic peptides from global protein quantification using iTRAQ, B) Representative MS/MS spectrum from glycosite-containing peptides from a CD63. iTRAQ reporter ion 114 and 115 represent repeated analyses of LNCap cells, and 116 reporter channel represents PC3 cells. *indicates iTRAQ modification, 0 indicates Carbamidomethylation of cysteine, ˜indicates oxidation on methionine, and lower case n indicates N-linked glycosylation site.

FIG. 10. Quantitative analysis of intact glycopeptides. The intact glycopeptides with a minimum of five MS/MS spectra matching with both high mannose and complex fucosylated glycans assigned to the same N-glycosites are presented here. VQPFN*VTQGK (lysosome associated membrane protein 2) (SEQ ID NO:5), VPYNVGPGFTGN*FSTQK (glutamate carboxypeptidase) (SEQ ID NO:6), YYN*YTLSINGK (glucosamine 6 sulfatase) (SEQ ID NO:7), HNN*DTQHIWESDSNEFSVIADPR (heat shock protein 90 B1) (SEQ ID NO:8), AGPN*GTLFVADAYK (adipocyte plasma membrane protein) (SEQ ID NO:1) and NLEKN*STKQEILAALEK (SEQ ID NO:2), TN*STFVQALVEHVKEECDR (SEQ ID NO:9), TN*STFVQALVEHVK (SEQ ID NO:10), LIDNN*KTEK (prosaposin) (SEQ ID NO:11), and DVVTAAGDMLKDN*ATEEEILVYLEK (SEQ ID NO:12). The y-axis represents the ratio observed between PC3/LNCaP cells using iTRAQ-based quantification. The number above each bar indicates the number of MS/MS spectra used to determine the glycopeptide ratio between PC3 and LNCaP cells.

FIG. 11. MS/MS spectra of glycosite-containing peptides (glycans were removed) and glycopeptides containing different glycans at the same glycosite. A) A MSMS spectrum of glycosite-containing peptide, AGPN*GTLFVADAYK (SEQ ID NO:1), identified by SPEG method after PNGaseF treatment. B) A MSMS spectrum of glycopeptide, AGPN*GTLFVADAYK (SEQ ID NO:1), containing HexNAc2Hex5. C) A MSMS spectrum of identified glycopeptide AGPN*GTLFVADAYK (SEQ ID NO:1) containing HexNAc 2 Hex5 Fuc1. The glycan fragments are the most plausible ions based on accurate mass. The peptide with same backbone was observed in all three spectra. The Oxonium ions represent the presence of the glycan structures. The reporter ion intensity represents decrease in high mannose structures and increase in fucosylation on the peptide AGPNGTLFVADAYK (SEQ ID NO:1) in PC3 cells (116) compared to LNCap cells (114,115). * (or #) indicates deamidation of N due to PNGaseF treatment.

FIG. 12. Graph showing gene expression.

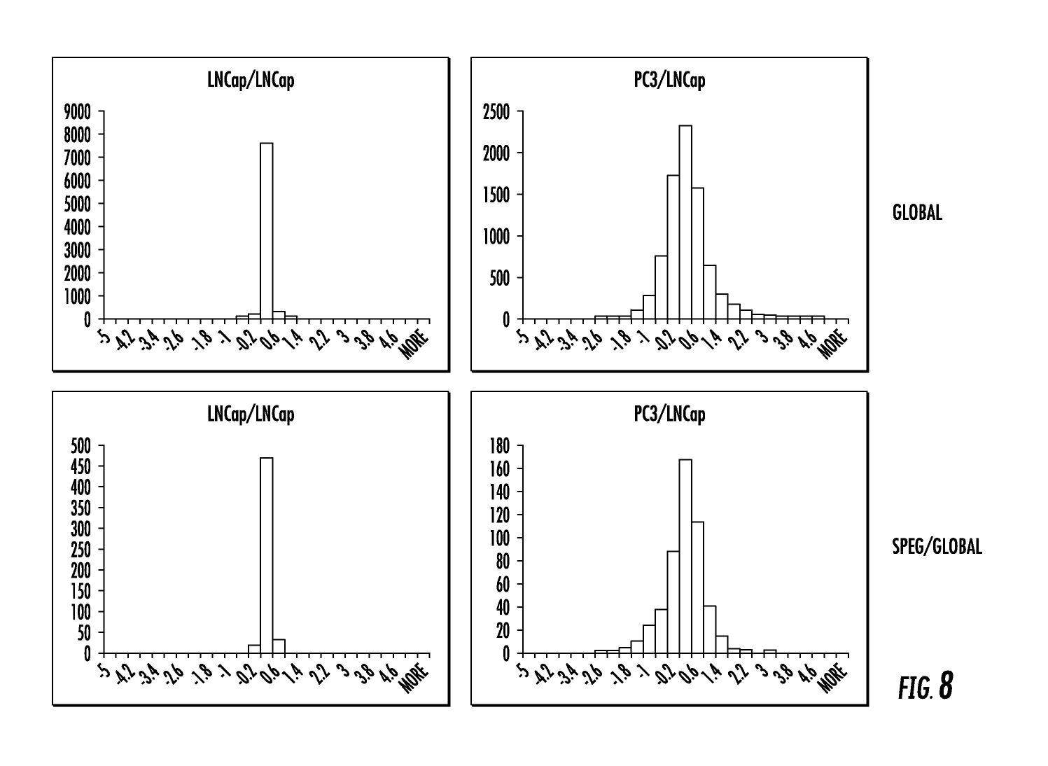

FIG. 13. FUT8 expression in additional Ad-independent cells.

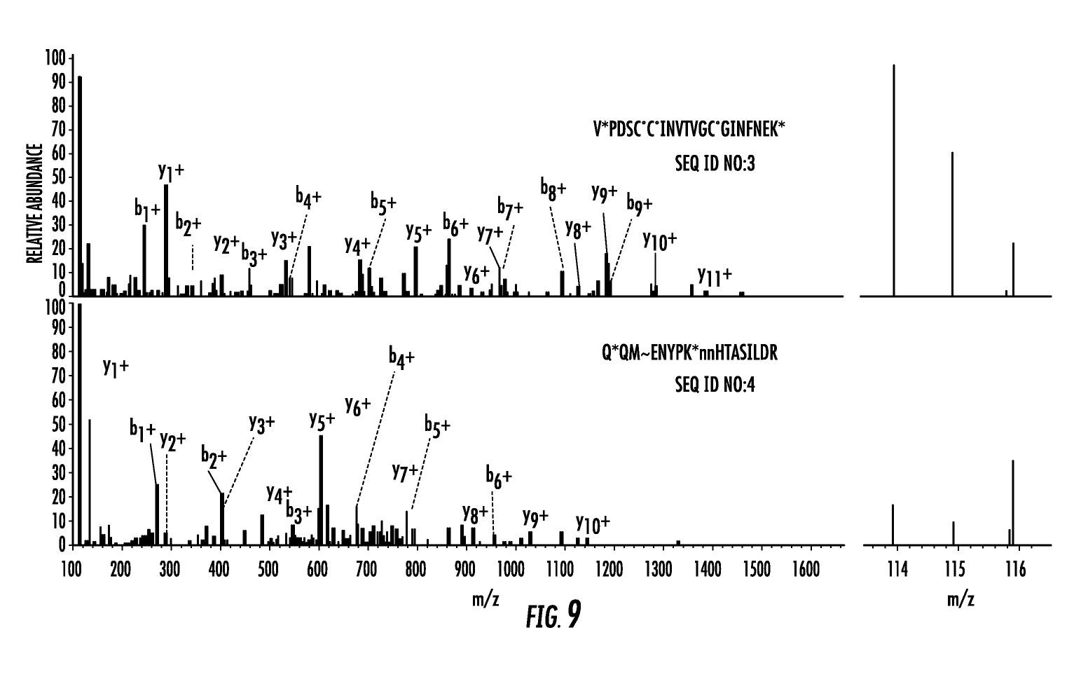

FIG. 14. Genomic, proteomic and glycoproteomic analyses of prostate cancer cells.

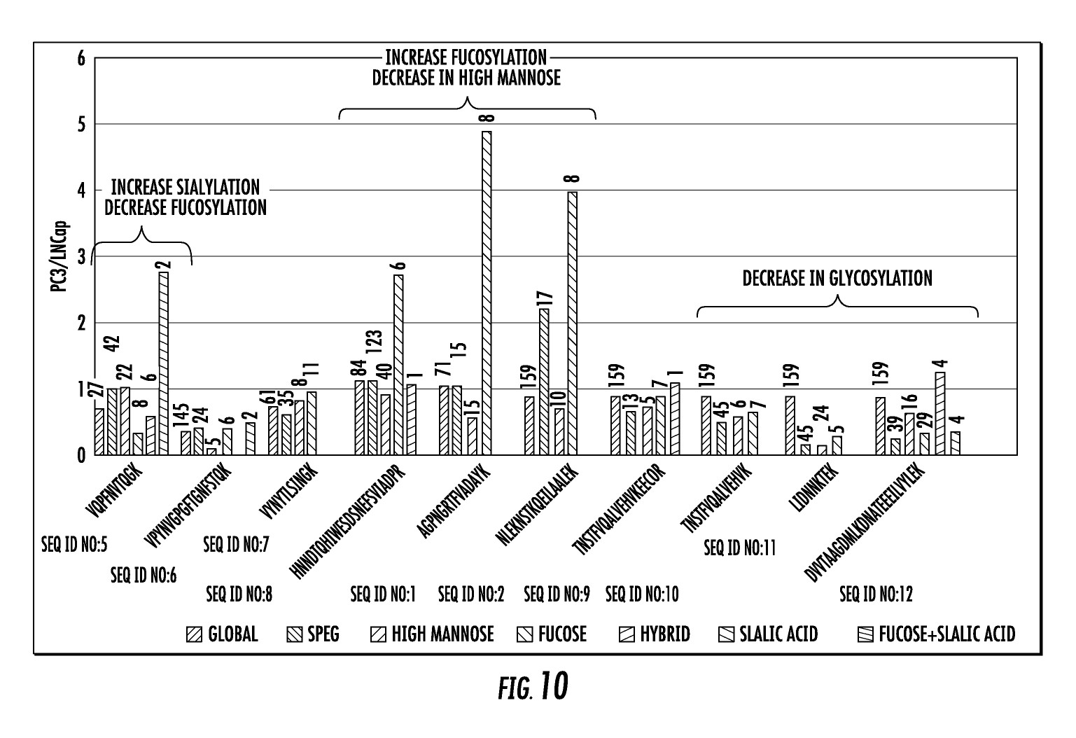

FIG. 15. Summary of LNCaP and PC3 cells in gene expression, protein level and glycoprotein level.

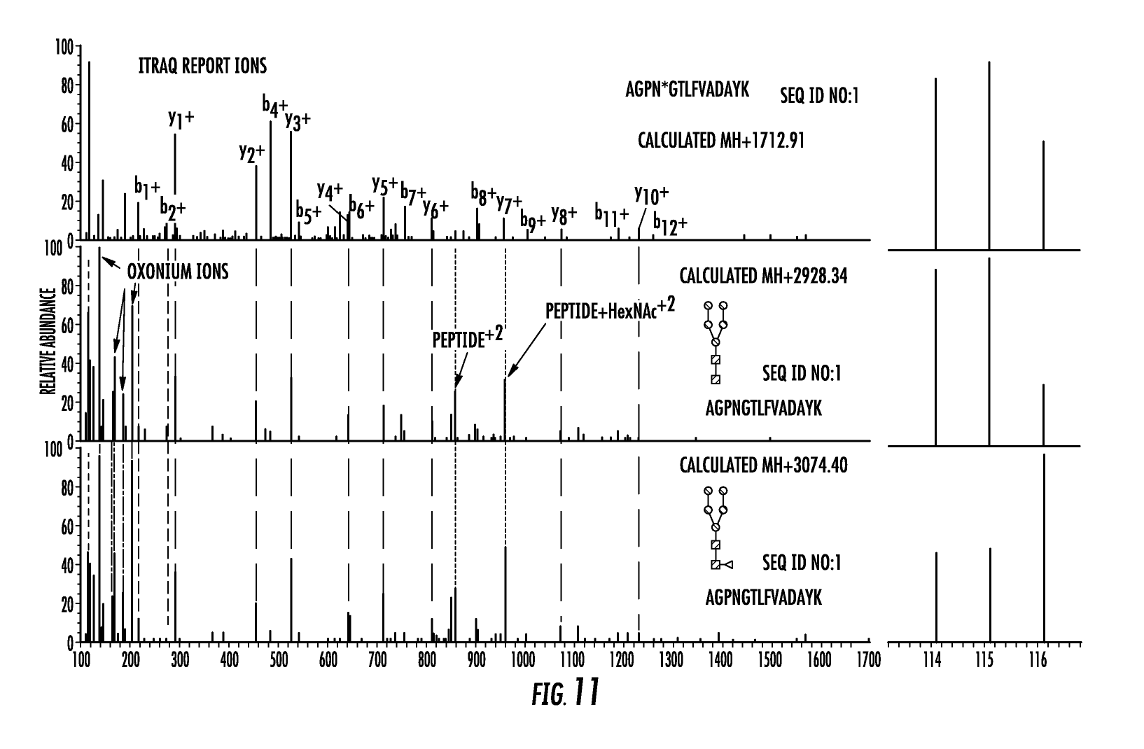

FIG. 16. FUT8 expression versus Gleason score.

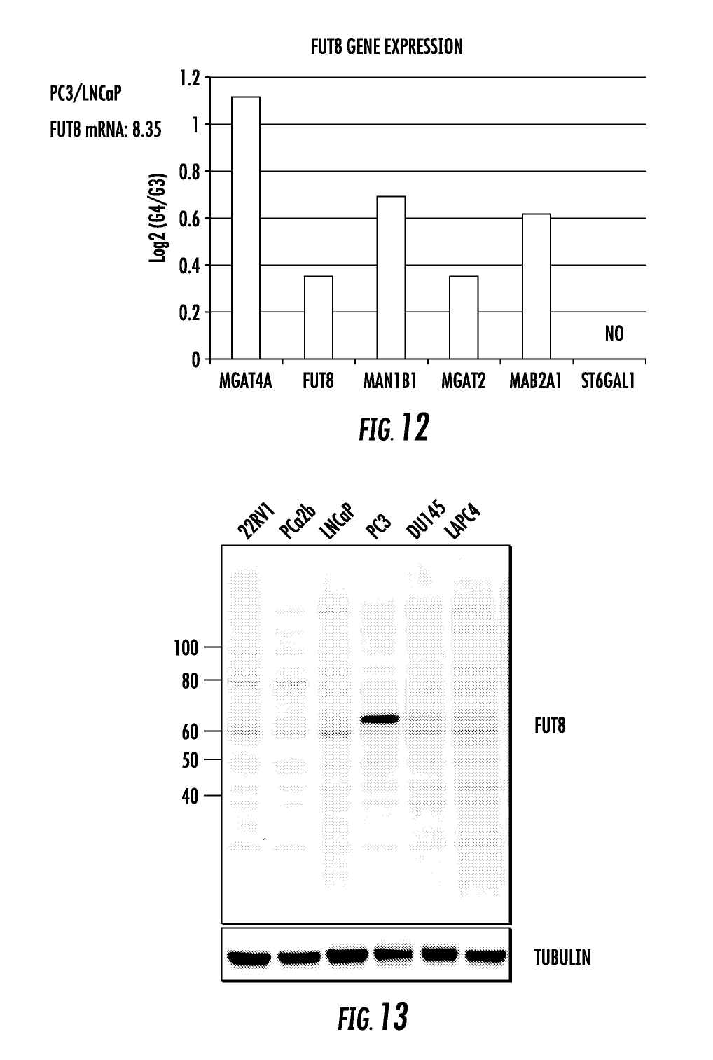

FIG. 17A. Immunohistochemistry of prostate cancer tissues using FUT8.

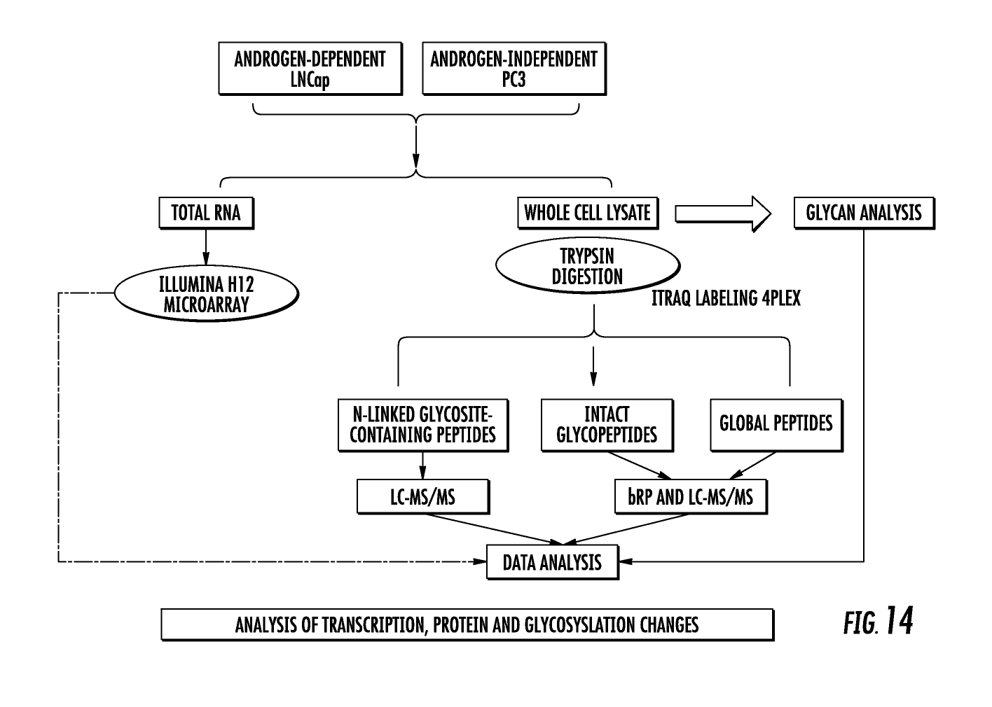

FIG. 17B. Summary of the 10 individual prostate cancer FUT8 immunohistochemistry.

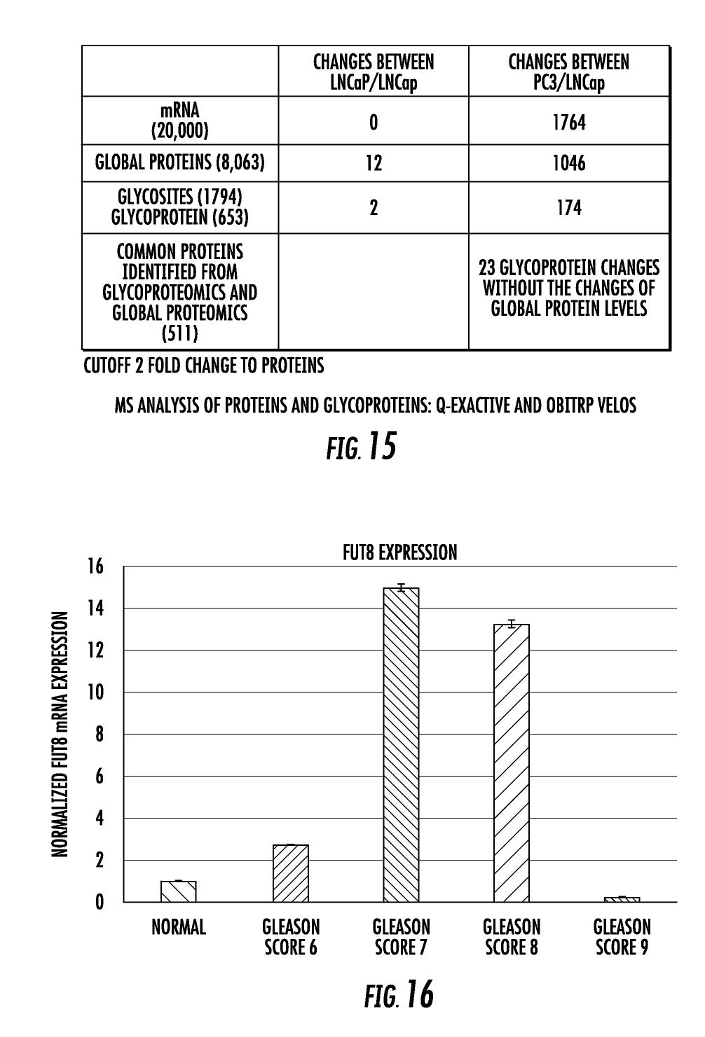

FIG. 18. QRT-PCR mRNA expression of FUT8.

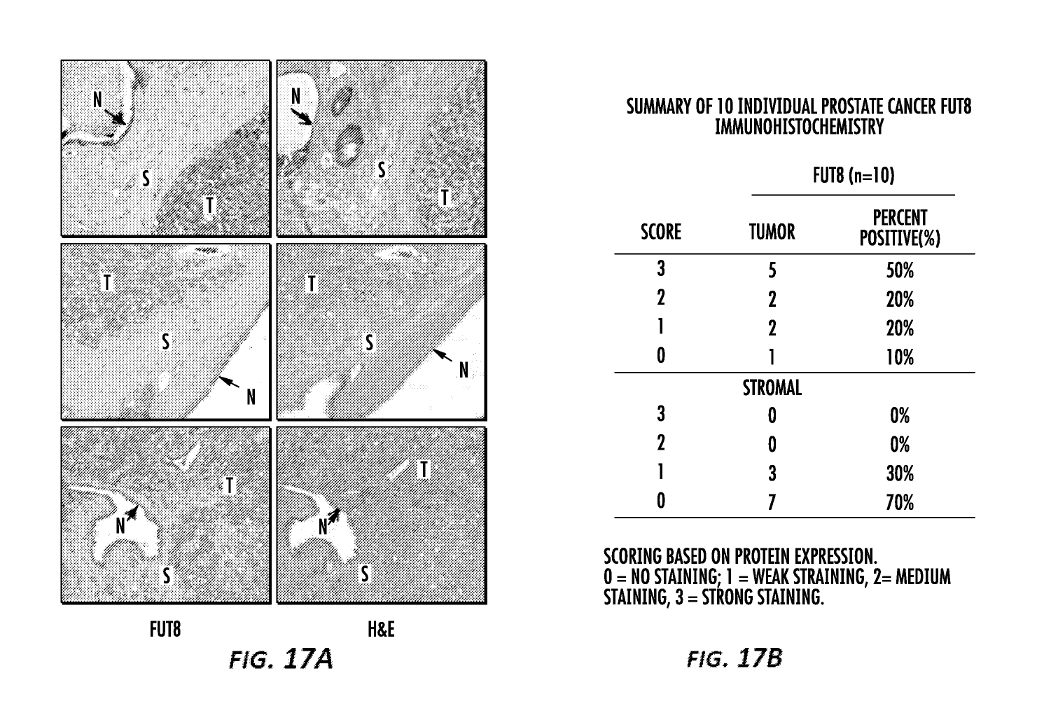

FIG. 19. Summary of LNCaP and LNCaP-FUT8 cells in protein expression.

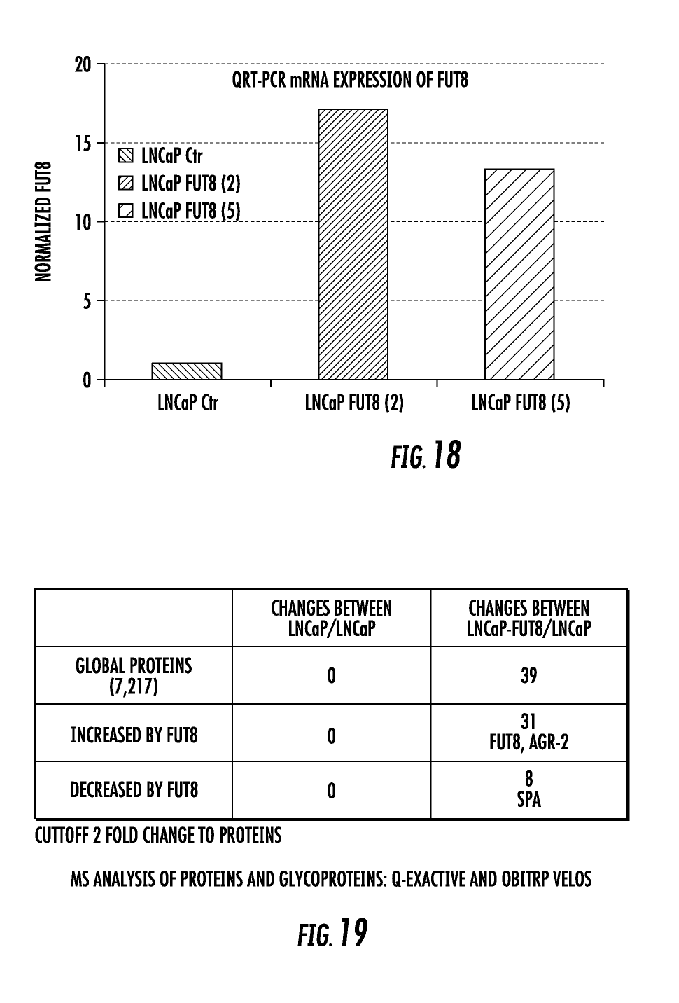

FIG. 20. High expression of FUT8 in low PSA level patient.

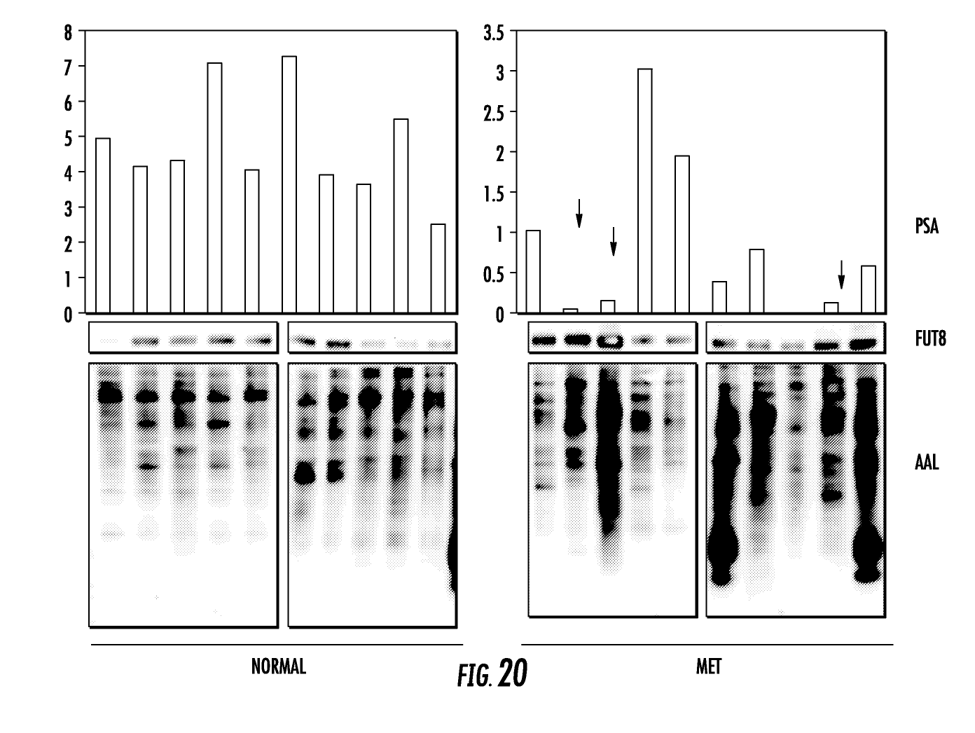

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

### I. DEFINITIONS

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes any fragment or derivative of any of the herein described antibodies.

As used herein, the term “antigen” is generally used in reference to any substance that is capable of reacting with an antibody. More specifically, as used herein, the term “antigen” refers to a biomarker described herein. An antigen can also refer to a synthetic peptide, polypeptide, protein or fragment of a polypeptide or protein, or other molecule which elicits an antibody response in a subject, or is recognized and bound by an antibody.

As used herein, the term “biomarker” refers to a molecule that is associated either quantitatively or qualitatively with a biological change. Examples of biomarkers include proteins, polypeptides, proteins or fragments of a polypeptide or protein; and polynucleotides, such as a gene product, RNA or RNA fragment. In certain embodiments, a “biomarker” means a compound that is differentially present (i.e., increased or decreased) in a biological sample from a subject or a group of subjects having a first phenotype (e.g., having a disease or condition) as compared to a biological sample from a subject or group of subjects having a second phenotype (e.g., not having the disease or condition or having a less severe version of the disease or condition). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using, for example, either Welch's T-test or Wilcoxon's rank-sum Test). Biomarker levels can be used, in conjunction with other parameters to distinguish aggressive prostate cancer from non-aggressive prostate cancer in a patient.

The term “one or more of” refers to combinations of various biomarker proteins. The term encompasses 1, 2, 3, 4, 5, 6, 7, 8, 9, 10, 11, 12, 13, 15, 16, 17, 18, 19, 20, 21, 22, 23, 24, 25, 26, 27, 28, 29, 30, 31, 32, 33, 34, 35, 36, 37, 38, 39, 40 . . . N, where “N” is the total number of biomarker proteins in the particular embodiment. The term also encompasses at least 1, at least 2, at least 3, at least 4, at least 5, at least 6, at least 7, at least 8, at least 9, at least 10, at least 11, at least 12, at least 13, at least 15, 16, 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40 . . . N. It is understood that the recitation of biomarkers herein includes the phrase “one or more of” the biomarkers and, in particular, includes the “at least 1, at least 2, at least 3” and so forth language in each recited embodiment of a biomarker panel.

As used herein, the term “comparing” refers to making an assessment of how the proportion, level or cellular localization of one or more biomarkers in a sample from a patient relates to the proportion, level or cellular localization of the corresponding one or more biomarkers in a standard, reference or control sample. For example, “comparing” may refer to assessing whether the proportion, level, or cellular localization of one or more biomarkers in a sample from a patient is the same as, more or less than, or different from the proportion, level, or cellular localization of the corresponding one or more biomarkers in standard, reference or control sample. More specifically, the term may refer to assessing whether the proportion, level, or cellular localization of one or more biomarkers in a sample from a patient is the same as, more or less than, different from or otherwise corresponds (or not) to the proportion, level, or cellular localization of predefined biomarker levels/ratios that correspond to, for example, a patient having aggressive prostate cancer, not having aggressive prostate cancer (e.g., non-aggressive prostate cancer or no cancer), is responding to treatment for aggressive prostate cancer, is not responding to treatment for aggressive prostate cancer, is/is not likely to respond to a particular aggressive prostate cancer treatment, or having/not having another disease or condition. In a specific embodiment, the term “comparing” refers to assessing whether the level of one or more biomarkers of the present invention in a sample from a patient is the same as, more or less than, different from other otherwise correspond (or not) to levels/ratios of the same biomarkers in a control sample (e.g., predefined levels/ratios that correlate to uninfected individuals, non-aggressive prostate cancer, standard aggressive prostate cancer levels/ratios, etc.).

In another embodiment, the term “comparing” refers to making an assessment of how the proportion, level or cellular localization of one or more biomarkers in a sample from a patient relates to the proportion, level or cellular localization of another biomarker in the same sample. For example, a ratio of one biomarker to another from the same patient sample can be compared. In another embodiment, a level of one biomarker in a sample (e.g., a post-translationally modified biomarker protein) can be compared to the level of the same biomarker (e.g., unmodified biomarker protein) in the sample. In a specific embodiment, the proportion of a fucosylated biomarker protein can be compared to the unmodified protein, both of which are measured in the same patient sample. Ratios of modified:unmodified biomarker proteins can be compared to other protein ratios in the same sample or to predefined reference or control ratios.

As used herein, the terms “identifies,” “indicates” or “correlates” (or “identifying,” “indicating” or “correlating,” or “identification,” “indication” or “correlation,” depending on the context) in reference to a parameter, e.g., a modulated proportion, level, or cellular localization in a sample from a patient, may mean that the patient has aggressive prostate cancer. In specific embodiments, the parameter may comprise the level of one or more biomarkers of the present invention. A particular set or pattern of the amounts of one or more biomarkers may identify the patient as having aggressive prostate cancer (i.e., correlates to a patient having aggressive prostate cancer). In other embodiments, a correlation could be that the ratio of a post-translationally modified protein (e.g., fucosylation) to the unmodified protein indicates (or a change in the ratio over time or as compared to a reference/control ratio) that the patient has aggressive prostate cancer. In specific embodiments, an indication could be the ratio of a fucosylated peptide to the non-fucosylated form, or any other combination in which a change in one peptide causes or is accompanied by a change in another.

In other embodiments, a particular set or pattern of the amounts of one or more biomarkers may identify the patient as being unaffected (i.e., indicates a patient does not have aggressive prostate cancer, a patient has non-aggressive prostate cancer, or a patient does not have cancer). In certain embodiments, “identifying,” “indicating,” or “correlating,” as used according to the present invention, may be by any linear or non-linear method of quantifying the relationship between levels/ratios of biomarkers to a standard, control or comparative value for the assessment of the diagnosis, prediction of aggressive prostate cancer or aggressive prostate cancer progression, assessment of efficacy of clinical treatment, identification of a patient that may respond to a particular treatment regime or pharmaceutical agent, monitoring of the progress of treatment, and in the context of a screening assay, for the identification of an anti-aggressive prostate cancer therapeutic.

The terms “patient,” “individual,” or “subject” are used interchangeably herein, and refer to a mammal, particularly, a human. The patient may have mild, intermediate or severe disease. The patient may be treatment naïve, responding to any form of treatment, or refractory. The patient may be an individual in need of treatment or in need of diagnosis based on particular symptoms or family history. In some cases, the terms may refer to treatment in experimental animals, in veterinary application, and in the development of animal models for disease, including, but not limited to, rodents including mice, rats, and hamsters; and primates.

The terms “measuring” and “determining” are used interchangeably throughout, and refer to methods which include obtaining a patient sample and/or detecting the level of a biomarker(s) in a sample. In one embodiment, the terms refer to obtaining a patient sample and detecting the level of one or more biomarkers in the sample. In another embodiment, the terms “measuring” and “determining” mean detecting the level of one or more biomarkers in a patient sample. Measuring can be accomplished by methods known in the art and those further described herein. The terms are also used interchangeably throughout with the term “detecting.”

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms of aggressive prostate cancer. Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cerebrospinal fluid, urine, saliva, stool and synovial fluid), solid tissue samples such as a biopsy specimen or tissue cultures or cells derived therefrom and the progeny thereof. In a specific embodiment, a sample comprises a blood sample. In another embodiment, a sample comprises a plasma sample. In yet another embodiment, a serum sample is used.

As used herein, the term “predetermined threshold value” of a biomarker refers to the level of the same biomarker in a corresponding control/normal sample or group of control/normal samples obtained from normal, or healthy, subjects, e.g., subjects who do not have a kidney disease, disorder or condition. Further, the term “altered level” of a biomarker in a sample refers to a level that is either below or above the predetermined threshold value for the same biomarker and thus encompasses either high (increased) or low (decreased) levels.

As used herein, the terms “binding agent specific for” or “binding agent that specifically binds” refers to an agent that binds to a biomarker and does not significantly bind to unrelated compounds. Examples of binding agents that can be effectively employed in the disclosed methods include, but are not limited to, lectins, proteins and antibodies, such as monoclonal or polyclonal antibodies, or antigen-binding fragments thereof, aptamers, etc. In certain embodiments, a binding agent binds a biomarker with an affinity constant of, for example, greater than or equal to about 1×10⁻⁶ M.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry. In certain embodiments, a sample comprises an optimal cutting temperature (OCT)-embedded frozen tissue sample.

The terms “specifically binds to,” “specific for,” and related grammatical variants refer to that binding which occurs between such paired species as enzyme/substrate, receptor/agonist, antibody/antigen, and lectin/carbohydrate which may be mediated by covalent or non-covalent interactions or a combination of covalent and non-covalent interactions. When the interaction of the two species produces a non-covalently bound complex, the binding which occurs is typically electrostatic, hydrogen-bonding, or the result of lipophilic interactions. Accordingly, “specific binding” occurs between a paired species where there is interaction between the two which produces a bound complex having the characteristics of an antibody/antigen or enzyme/substrate interaction. In particular, the specific binding is characterized by the binding of one member of a pair to a particular species and to no other species within the family of compounds to which the corresponding member of the binding member belongs. Thus, for example, an antibody typically binds to a single epitope and to no other epitope within the family of proteins. In some embodiments, specific binding between an antigen and an antibody will have a binding affinity of at least 10⁻⁶ M. In other embodiments, the antigen and antibody will bind with affinities of at least 10⁻⁷ M, 10⁻⁸ M to 10⁻⁹ M, 10⁻¹⁰ M, 10⁻¹¹ M, or 10⁻¹² M.

Various methodologies of the instant invention include a step that involves comparing a value, level, feature, characteristic, property, etc. to a “suitable control,” referred to interchangeably herein as an “appropriate control,” a “control sample” or a “reference.” A “suitable control,” “appropriate control,” “control sample” or a “reference” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed for levels/ratios in a sample from an unaffected individual (UI) or a normal control individual (NC) (both terms are used interchangeably herein). In another embodiment, a “suitable control” or “appropriate control” is a value, level, feature, characteristic, property, ratio, etc. determined prior to performing a therapy (e.g., aggressive prostate cancer treatment) on a patient. In yet another embodiment, a transcription rate, mRNA level, translation rate, protein level/ratio, biological activity, cellular characteristic or property, genotype, phenotype, etc., can be determined prior to, during, or after administering a therapy into a cell, organ, or patient. In a further embodiment, a “suitable control,” “appropriate control” or a “reference” is a predefined value, level, feature, characteristic, property, ratio, etc. A “suitable control” can be a profile or pattern of levels/ratios of one or more biomarkers of the present invention that correlates to aggressive prostate cancer, to which a patient sample can be compared. The patient sample can also be compared to a negative control, i.e., a profile that correlates to having non-aggressive prostate cancer. Such reference levels may also be tailored to specific techniques that are used to measure levels of biomarkers in biological samples (e.g., LC-MS, GC-MS, ELISA, PCR, etc.), where the levels of biomarkers may differ based on the specific technique that is used.

### II. DETECTION OF AGGRESSIVE PROSTATE CANCER BIOMARKERS

A. Detection by Immunoassay

In one aspect, the biomarkers of the present invention may be detected and/or measured by immunoassay Immunoassay requires biospecific capture reagents, such as antibodies, to capture the biomarkers. Many antibodies are available commercially. Antibodies also can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well-known in the art. Biospecific capture reagents useful in an immunoassay can also include lectins. The biospecific capture reagents can, in some embodiments, bind all forms of the biomarker, e.g., PSA and its post-translationally modified forms (fucosylated form). In other embodiments, the biospecific capture reagents bind the specific biomarker (e.g., fucosylated PSA) and not similar forms thereof.

The present invention contemplates traditional immunoassays including, for example, sandwich immunoassays including ELISA or fluorescence-based immunoassays, immunoblots, Western Blots (WB), as well as other enzyme immunoassays. Nephelometry is an assay performed in liquid phase, in which antibodies are in solution. Binding of the antigen to the antibody results in changes in absorbance, which is measured. In a SELDI-based immunoassay, a biospecific capture reagent for the biomarker is attached to the surface of an MS probe, such as a pre-activated protein chip array. The biomarker is then specifically captured on the biochip through this reagent, and the captured biomarker is detected by mass spectrometry.

Although antibodies are useful because of their extensive characterization, any other suitable agent (e.g., a lectin, peptide, aptamer or a small organic molecule) that specifically binds a biomarker of the present invention is optionally used in place of the antibody in the above described immunoassays. Exemplary lectins are described throughout the specification. Furthermore, an exemplary aptamer that specifically binds all a biomarkers (and, in certain embodiments, its post-translationally modified forms) might be used. Aptamers are nucleic acid-based molecules that bind specific ligands. Methods for making aptamers with a particular binding specificity are known as detailed in U.S. Pat. No. 5,475,096; U.S. Pat. No. 5,670,637; U.S. Pat. No. 5,696,249; U.S. Pat. No. 5,270,163; U.S. Pat. No. 5,707,796; U.S. Pat. No. 5,595,877; U.S. Pat. No. 5,660,985; U.S. Pat. No. 5,567,588; U.S. Pat. No. 5,683,867; U.S. Pat. No. 5,637,459; and U.S. Pat. No. 6,011,020.

In certain embodiments, the levels of the biomarkers employed herein are quantified by immunoassay, such as enzyme-linked immunoassay (ELISA) technology. In specific embodiments, the levels of expression of the biomarkers are determined by contacting the biological sample with antibodies, or antigen binding fragments thereof, that selectively bind to the biomarkers; and detecting binding of the antibodies, or antigen binding fragments thereof, to the biomarkers. In certain embodiments, the binding agents employed in the disclosed methods and compositions are labeled with a detectable moiety. For ease of reference, the term antibody is used in describing binding agents or capture molecules. However, it is understood that reference to an antibody in the context of describing an exemplary binding agent in the methods of the present invention also includes reference to other binding agents including, but not limited to lectins.

Furthermore, the level of a biomarker in a sample can be assayed by contacting the biological sample with an antibody, or antigen binding fragment thereof, that selectively binds to the target biomarker (referred to as a capture molecule or antibody or a binding agent), and detecting the binding of the antibody, or antigen-binding fragment thereof, to the biomarker. The detection can be performed using a second antibody to bind to the capture antibody complexed with its target biomarker. Kits for the detection of biomarkers as described herein can include pre-coated strip plates, biotinylated secondary antibody, standards, controls, buffers, streptavidin-horse radish peroxidise (HRP), tetramethyl benzidine (TMB), stop reagents, and detailed instructions for carrying out the tests including performing standards.

The present disclosure also provides methods in which the levels of the biomarkers in a biological sample are determined simultaneously. For example, in one embodiment, methods are provided that comprise: (a) contacting a biological sample obtained from the subject with a plurality of binding agents that selectively bind to a plurality of biomarkers disclosed herein for a period of time sufficient to form binding agent-biomarker complexes; (b) detecting binding of the binding agents to the plurality of biomarkers, thereby determining the levels of the biomarkers in the biological sample; and (c) comparing the levels of the plurality of biomarkers in the biological sample with predetermined threshold values, wherein levels of at least one of the plurality of biomarkers above/below the predetermined threshold values can be used to distinguish aggressive prostate cancer. Examples of binding agents that can be effectively employed in such methods include, but are not limited to, antibodies or antigen-binding fragments thereof, lectins, aptamers and the like.

In a further aspect, the present disclosure provides compositions that can be employed in the disclosed methods. In certain embodiments, such compositions a solid substrate and a plurality of binding agents immobilized on the substrate, wherein each of the binding agents is immobilized at a different, indexable, location on the substrate and the binding agents selectively bind to a plurality of biomarkers disclosed herein. In a specific embodiment, the locations are pre-determined. In other embodiments, kits are provided that comprise such compositions. In certain embodiments, the plurality of biomarkers includes one or more of the biomarkers described herein including PSA.

In a related aspect, methods for distinguishing aggressive prostate cancer from non-aggressive prostate cancer in a subject are provided, such methods comprising: (a) contacting a biological sample obtained from the subject with a composition disclosed herein for a period of time sufficient to form binding agent-biomarker complexes; (b) detecting binding of the binding agents to a plurality of biomarkers, thereby determining the levels of biomarkers in the biological sample; and (c) comparing the levels of biomarkers in the biological sample with predetermined threshold values, wherein levels of expression of at least one of the plurality of biomarkers above/below the predetermined threshold values can be used to distinguish aggressive prostate cancer.

In specific embodiments, the assay performed on the biological sample can comprise contacting the biological sample with one or more capture agents (e.g., antibodies, lectins, peptides, aptamer, etc., combinations thereof) to form a biomarker:capture agent complex. The complexes can then be detected and/or quantified.

In one method, a first capture molecule or binding agent, such as an antibody that specifically binds the biomarker of interest, is immobilized on a suitable solid phase substrate or carrier. The test biological sample is then contacted with the capture antibody and incubated for a desired period of time. After washing to remove unbound material, a second, detection, antibody that binds to a different, non-overlapping, epitope on the biomarker is then used to detect binding of the biomarker to the capture antibody. The detection antibody is preferably conjugated, either directly or indirectly, to a detectable moiety. Examples of detectable moieties that can be employed in such methods include, but are not limited to, cheminescent and luminescent agents; fluorophores such as fluorescein, rhodamine and eosin; radioisotopes; colorimetric agents; and enzyme-substrate labels, such as biotin.

In a more specific embodiment, a biotinylated lectin that specifically binds a biomarker (e.g., fucosylated PSA) can be added to a patient sample and a streptavidin labeled fluorescent marker that binds the biotinylated lectin bound to the biomarker is then added, and the biomarker is detected.

In another embodiment, the assay is a competitive binding assay, wherein labeled biomarker is used in place of the labeled detection antibody, and the labeled biomarker and any unlabeled biomarker present in the test sample compete for binding to the capture antibody. The amount of biomarker bound to the capture antibody can be determined based on the proportion of labeled biomarker detected.

Solid phase substrates, or carriers, that can be effectively employed in such assays are well known to those of skill in the art and include, for example, 96 well microtiter plates, glass, paper, chips and microporous membranes constructed, for example, of nitrocellulose, nylon, polyvinylidene difluoride, polyester, cellulose acetate, mixed cellulose esters and polycarbonate. Suitable microporous membranes include, for example, those described in US Patent Application Publication no. US 2010/0093557 A1. Methods for the automation of immunoassays are well known in the art and include, for example, those described in U.S. Pat. Nos. 5,885,530, 4,981,785, 6,159,750 and 5,358,691.

The presence of several different biomarkers in a test sample can be detected simultaneously using a multiplex assay, such as a multiplex ELISA. Multiplex assays offer the advantages of high throughput, a small volume of sample being required, and the ability to detect different proteins across a board dynamic range of concentrations.

In certain embodiments, such methods employ an array, wherein multiple binding agents (for example capture antibodies) specific for multiple biomarkers are immobilized on a substrate, such as a membrane, with each capture agent being positioned at a specific, pre-determined, location on the substrate. Methods for performing assays employing such arrays include those described, for example, in US Patent Application Publication nos. US2010/0093557A1 and US2010/0190656A1, the disclosures of which are hereby specifically incorporated by reference.

Multiplex arrays in several different formats based on the utilization of, for example, flow cytometry, chemiluminescence or electron-chemiluminesence technology, are well known in the art. Flow cytometric multiplex arrays, also known as bead-based multiplex arrays, include the Cytometric Bead Array (CBA) system from BD Biosciences (Bedford, Mass.) and multi-analyte profiling (xMAP®) technology from Luminex Corp. (Austin, Tex.), both of which employ bead sets which are distinguishable by flow cytometry. Each bead set is coated with a specific capture antibody. Fluorescence or streptavidin-labeled detection antibodies bind to specific capture antibody-biomarker complexes formed on the bead set. Multiple biomarkers can be recognized and measured by differences in the bead sets, with chromogenic or fluorogenic emissions being detected using flow cytometric analysis. In an alternative format, a multiplex ELISA from Quansys Biosciences (Logan, Utah) coats multiple specific capture antibodies at multiple spots (one antibody at one spot) in the same well on a 96-well microtiter plate. Chemiluminescence technology is then used to detect multiple biomarkers at the corresponding spots on the plate.

B. Detection by Mass Spectrometry

In another aspect, the biomarkers of the present invention may be detected by mass spectrometry, a method that employs a mass spectrometer to detect gas phase ions. Examples of mass spectrometers are time-of-flight, magnetic sector, quadrupole filter, ion trap, ion cyclotron resonance, Orbitrap, hybrids or combinations of the foregoing, and the like.

In particular embodiments, the biomarkers of the present invention are measured/detected using selected reaction monitoring (SRM) mass spectrometry techniques. Selected reaction monitoring (SRM) is a non-scanning mass spectrometry technique, performed on triple quadrupole-like instruments and in which collision-induced dissociation is used as a means to increase selectivity. In SRM experiments two mass analyzers are used as static mass filters, to monitor a particular fragment ion of a selected precursor ion. The specific pair of mass-over-charge (m/z) values associated to the precursor and fragment ions selected is referred to as a “transition” and can be written as parent m/z→fragment m/z (e.g. 673.5→534.3). Unlike common MS based proteomics, no mass spectra are recorded in a SRM analysis. Instead, the detector acts as counting device for the ions matching the selected transition thereby returning an intensity distribution over time. Multiple SRM transitions can be measured within the same experiment on the chromatographic time scale by rapidly toggling between the different precursor/fragment pairs (sometimes called multiple reaction monitoring, MRM). Typically, the triple quadrupole instrument cycles through a series of transitions and records the signal of each transition as a function of the elution time. The method allows for additional selectivity by monitoring the chromatographic coelution of multiple transitions for a given analyte. The terms SRM/MRM are occasionally used also to describe experiments conducted in mass spectrometers other than triple quadrupoles (e.g. in trapping instruments) where upon fragmentation of a specific precursor ion a narrow mass range is scanned in MS2 mode, centered on a fragment ion specific to the precursor of interest or in general in experiments where fragmentation in the collision cell is used as a means to increase selectivity. In this application, the terms SRM and MRM or also SRM/MRM can be used interchangeably, since they both refer to the same mass spectrometer operating principle. As a matter of clarity, the term MRM can be used throughout the text, but the term includes both SRM and MRM, as well as any analogous technique, such as e.g. highly-selective reaction monitoring, hSRM, LC-SRM or any other SRM/MRM-like or SRM/MRM-mimicking approaches performed on any type of mass spectrometer and/or, in which the peptides are fragmented using any other fragmentation method such as e.g. CAD (collision-activated dissociation (also known as CID or collision-induced dissociation), HCD (higher energy CID), ECD (electron capture dissociation), PD (photodissociation) or ETD (electron transfer dissociation).

In another specific embodiment, the mass spectrometric method comprises matrix assisted laser desorption/ionization time-of-flight (MALDI-TOF MS or MALDI-TOF). In another embodiment, method comprises MALDI-TOF tandem mass spectrometry (MALDI-TOF MS/MS). In yet another embodiment, mass spectrometry can be combined with another appropriate method(s) as may be contemplated by one of ordinary skill in the art. For example, MALDI-TOF can be utilized with trypsin digestion and tandem mass spectrometry as described herein.

In an alternative embodiment, the mass spectrometric technique comprises surface enhanced laser desorption and ionization or “SELDI,” as described, for example, in U.S. Pat. No. 6,225,047 and U.S. Pat. No. 5,719,060. Briefly, SELDI refers to a method of desorption/ionization gas phase ion spectrometry (e.g. mass spectrometry) in which an analyte (here, one or more of the biomarkers) is captured on the surface of a SELDI mass spectrometry probe. There are several versions of SELDI that may be utilized including, but not limited to, Affinity Capture Mass Spectrometry (also called Surface-Enhanced Affinity Capture (SEAC)), and Surface-Enhanced Neat Desorption (SEND) which involves the use of probes comprising energy absorbing molecules that are chemically bound to the probe surface (SEND probe). Another SELDI method is called Surface-Enhanced Photolabile Attachment and Release (SEPAR), which involves the use of probes having moieties attached to the surface that can covalently bind an analyte, and then release the analyte through breaking a photolabile bond in the moiety after exposure to light, e.g., to laser light (see, U.S. Pat. No. 5,719,060). SEPAR and other forms of SELDI are readily adapted to detecting a biomarker or biomarker panel, pursuant to the present invention.

In another mass spectrometry method, the biomarkers can be first captured on a chromatographic resin having chromatographic properties that bind the biomarkers. For example, one could capture the biomarkers on a cation exchange resin, such as CM Ceramic HyperD F resin, wash the resin, elute the biomarkers and detect by MALDI. Alternatively, this method could be preceded by fractionating the sample on an anion exchange resin before application to the cation exchange resin. In another alternative, one could fractionate on an anion exchange resin and detect by MALDI directly. In yet another method, one could capture the biomarkers on an immuno-chromatographic resin that comprises antibodies that bind the biomarkers, wash the resin to remove unbound material, elute the biomarkers from the resin and detect the eluted biomarkers by MALDI or by SELDI.

C. Detection by Electrochemicaluminescent Assay

In several embodiments, the biomarker biomarkers of the present invention may be detected by means of an electrochemicaluminescent assay developed by Meso Scale Discovery (Gaithersrburg, Md.). Electrochemiluminescence detection uses labels that emit light when electrochemically stimulated. Background signals are minimal because the stimulation mechanism (electricity) is decoupled from the signal (light). Labels are stable, non-radioactive and offer a choice of convenient coupling chemistries. They emit light at ˜620 nm, eliminating problems with color quenching. See U.S. Pat. No. 7,497,997; U.S. Pat. No. 7,491,540; U.S. Pat. No. 7,288,410; U.S. Pat. No. 7,036,946; U.S. Pat. No. 7,052,861; U.S. Pat. No. 6,977,722; U.S. Pat. No. 6,919,173; U.S. Pat. No. 6,673,533; U.S. Pat. No. 6,413,783; U.S. Pat. No. 6,362,011; U.S. Pat. No. 6,319,670; U.S. Pat. No. 6,207,369; U.S. Pat. No. 6,140,045; U.S. Pat. No. 6,090,545; and U.S. Pat. No. 5,866,434. See also U.S. Patent Applications Publication No. 2009/0170121; No. 2009/006339; No. 2009/0065357; No. 2006/0172340; No. 2006/0019319; No. 2005/0142033; No. 2005/0052646; No. 2004/0022677; No. 2003/0124572; No. 2003/0113713; No. 2003/0003460; No. 2002/0137234; No. 2002/0086335; and No. 2001/0021534.

D. Other Methods for Detecting Biomarkers

The biomarkers of the present invention can be detected by other suitable methods. Detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Furthermore, a sample may also be analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. Protein biochips are biochips adapted for the capture of polypeptides. Many protein biochips are described in the art. These include, for example, protein biochips produced by Ciphergen Biosystems, Inc. (Fremont, Calif.), Invitrogen Corp. (Carlsbad, Calif.), Affymetrix, Inc. (Fremong, Calif.), Zyomyx (Hayward, Calif.), R&D Systems, Inc. (Minneapolis, Minn.), Biacore (Uppsala, Sweden) and Procognia (Berkshire, UK). Examples of such protein biochips are described in the following patents or published patent applications: U.S. Pat. No. 6,537,749; U.S. Pat. No. 6,329,209; U.S. Pat. No. 6,225,047; U.S. Pat. No. 5,242,828; PCT International Publication No. WO 00/56934; and PCT International Publication No. WO 03/048768.

### III. DETERMINATION OF A PATIENT'S AGGRESSIVE PROSTATE CANCER STATUS

The biomarkers of the present invention can be used in diagnostic tests, like a multiplex assay, to assess, determine, and/or qualify (used interchangeably herein) aggressive prostate cancer status in a patient. The phrase “aggressive prostate cancer status” includes any distinguishable manifestation of the condition, including not having aggressive prostate cancer. For example, aggressive prostate cancer status includes, without limitation, the presence or absence of aggressive prostate cancer in a patient, the risk of developing aggressive prostate cancer, the stage or severity of aggressive prostate cancer, the progress of aggressive prostate cancer (e.g., progress of aggressive prostate cancer over time) and the effectiveness or response to treatment of aggressive prostate cancer (e.g., clinical follow up and surveillance of aggressive prostate cancer after treatment). Based on this status, further procedures may be indicated, including additional diagnostic tests or therapeutic procedures or regimens.

A. Biomarker Panels

The present invention relates to the use of biomarkers to diagnose aggressive prostate cancer. More specifically, the biomarkers of the present invention can be used in diagnostic tests to determine, qualify, and/or assess aggressive prostate cancer or status, for example, to diagnose aggressive prostate cancer, in an individual, subject or patient. In particular embodiments, aggressive prostate cancer status can include determining a patient's aggressive prostate cancer status, for example, to diagnose aggressive prostate cancer, in an individual, subject or patient. More specifically, the biomarkers to be detected in diagnosing aggressive prostate cancer include PSA, UDP-glucuronosyltransferase, 2′-5′-oligoadenylate synthase-like protein, alpha-(1,6)-fucosyltransferase, annexin A1, G-protein coupled receptor 126, anterior gradient protein 2 homolog, calcium/calmodulin-dependent protein kinase II inhibitor 1, desmocollin-2 isoform Dsc2a preproprotein, interferon-induced protein with tetratricopeptide repeats 2, matrix metalloproteinase-15 preproprotein, interferon-induced protein with tetratricopeptide repeats 1, thymidylate synthase, interferon-induced protein with tetratricopeptide repeats 3, histone chaperone ASF1B, cyclin-dependent kinases regulatory subunit 2, nucleolar and spindle-associated protein 1, membrane associated phospholipase A2, ubiquitin-conjugating enzyme E2 C, solute carrier family 22 member 3, G2/mitotic-specific cyclin-B1, paternally-expressed gene 3 protein, PCNA-associated factor, paternally-expressed gene 3 protein, redox-regulatory protein FAM213A, kinesin family member 20A, cytosolic thymidine kinase, protein FAM83D, importin subunit alpha-1, transferrin receptor protein 1, hyaluronan mediated motility receptor, lymphokine-activated killer T-cell-originated protein kinase, transmembrane prostate androgen-induced protein, kallikrein-2 isoform 2 preproprotein, lipid phosphate phosphohydrolase 3, heparan-sulfate 6-O-sulfotransferase 1, prostatic acid phosphatase isoform PAP, nuclear RNA export factor 2, protein POF1B. The foregoing biomarkers and combinations thereof can include post-translationally modified forms and unmodified forms. Other biomarkers known in the relevant art may be used in combination with the biomarkers described herein including, but not limited to, sTIE-2, sVEGFR-1, fucosylated TIMP-1, fucosylated DPP-4, and FUT8, cathepsin-L (CTSL), periostin, microfibrillar-associated protein 4 (MFAP4), collagen XII, neprilysin, clusterin, neutrophil gelatinase associated lipocalin (NGAL), epithelial cell activating molecule (EpCAM), prostate specific antigen (PSA), membrane metallo-endopeptidase (MME) and asporin (ASPN). See, e.g., WO2013/116331; and WO2012/129408. FUT8 overexpression can also be used as a biomarker.

The power of a diagnostic test to correctly predict status is commonly measured as the sensitivity of the assay, the specificity of the assay or the area under a receiver operated characteristic (“ROC”) curve. Sensitivity is the percentage of true positives that are predicted by a test to be positive, while specificity is the percentage of true negatives that are predicted by a test to be negative. An ROC curve provides the sensitivity of a test as a function of 1-specificity. The greater the area under the ROC curve, the more powerful the predictive value of the test. Other useful measures of the utility of a test are positive predictive value and negative predictive value. Positive predictive value is the percentage of people who test positive that are actually positive. Negative predictive value is the percentage of people who test negative that are actually negative.

In particular embodiments, the biomarker panels of the present invention may show a statistical difference in different aggressive prostate cancer statuses of at least p<0.05, p<10⁻², p<10⁻³, p<10⁻⁴ or p<10⁻⁵. Diagnostic tests that use these biomarkers may show an ROC of at least 0.6, at least about 0.7, at least about 0.8, or at least about 0.9.

The biomarkers can be differentially present in UI (NC or non-aggressive prostate cancer) and aggressive prostate cancer, and, therefore, are useful in aiding in the determination of aggressive prostate cancer status. In certain embodiments, the biomarkers are measured in a patient sample using the methods described herein and compared, for example, to predefined biomarker levels/ratios and correlated to aggressive prostate cancer status. In particular embodiments, the measurement(s) may then be compared with a relevant diagnostic amount(s), cut-off(s), or multivariate model scores that distinguish a positive aggressive prostate cancer status from a negative aggressive prostate cancer status. The diagnostic amount(s) represents a measured amount of a biomarker(s) above which or below which a patient is classified as having a particular aggressive prostate cancer status. For example, if the biomarker(s) is/are up-regulated compared to normal (e.g., no cancer or non-aggressive prostate cancer) during aggressive prostate cancer, then a measured amount(s) above the diagnostic cutoff(s) provides a diagnosis of aggressive prostate cancer. Alternatively, if the biomarker(s) is/are down-regulated during aggressive prostate cancer, then a measured amount(s) at or below the diagnostic cutoff(s) provides a diagnosis of non-aggressive prostate cancer. The opposite may hold true as well (i.e., expression of the biomarker is lower/downregulated in aggressive prostate cancer vs. no cancer or non-aggressive prostate cancer) As is well understood in the art, by adjusting the particular diagnostic cut-off(s) used in an assay, one can increase sensitivity or specificity of the diagnostic assay depending on the preference of the diagnostician. In particular embodiments, the particular diagnostic cut-off can be determined, for example, by measuring the amount of biomarkers in a statistically significant number of samples from patients with the different aggressive prostate cancer statuses, and drawing the cut-off to suit the desired levels of specificity and sensitivity.

In other embodiments, ratios of post-translationally modified biomarkers (e.g., fucosylation, glycosylation, citrullination, oxidation, methylation, phosphorylation, cysteinylation s-nitrosation, s-glutathyolation, or a combination thereof) to the corresponding unmodified biomarkers are useful in aiding in the determination of aggressive prostate cancer status. In certain embodiments, the biomarker ratios are indicative of diagnosis. In other embodiments, a biomarker ratio can be compared to another biomarker ration in the same sample or to a set of biomarker ratios from a control or reference sample. In further embodiments, the amount(s) of a post-translationally modified biomarker(s) (e.g., fucosylated) can be compared to a reference or control sample (predefined amounts correlating to aggressive prostate cancer, non-aggressive prostate cancer, no cancer, and the like).

Indeed, as the skilled artisan will appreciate there are many ways to use the measurements of two or more biomarkers in order to improve the diagnostic question under investigation. In a quite simple, but nonetheless often effective approach, a positive result is assumed if a sample is positive for at least one of the markers investigated.

Furthermore, in certain embodiments, the values measured for markers of a biomarker panel are mathematically combined and the combined value is correlated to the underlying diagnostic question. Biomarker values may be combined by any appropriate state of the art mathematical method. Well-known mathematical methods for correlating a marker combination to a disease status employ methods like discriminant analysis (DA) (e.g., linear-, quadratic-, regularized-DA), Discriminant Functional Analysis (DFA), Kernel Methods (e.g., SVM), Multidimensional Scaling (MDS), Nonparametric Methods (e.g., k-Nearest-Neighbor Classifiers), PLS (Partial Least Squares), Tree-Based Methods (e.g., Logic Regression, CART, Random Forest Methods, Boosting/Bagging Methods), Generalized Linear Models (e.g., Logistic Regression), Principal Components based Methods (e.g., SIMCA), Generalized Additive Models, Fuzzy Logic based Methods, Neural Networks and Genetic Algorithms based Methods. The skilled artisan will have no problem in selecting an appropriate method to evaluate a biomarker combination of the present invention. In one embodiment, the method used in a correlating a biomarker combination of the present invention, e.g. to diagnose aggressive prostate cancer, is selected from DA (e.g., Linear-, Quadratic-, Regularized Discriminant Analysis), DFA, Kernel Methods (e.g., SVM), MDS, Nonparametric Methods (e.g., k-Nearest-Neighbor Classifiers), PLS (Partial Least Squares), Tree-Based Methods (e.g., Logic Regression, CART, Random Forest Methods, Boosting Methods), or Generalized Linear Models (e.g., Logistic Regression), and Principal Components Analysis. Details relating to these statistical methods are found in the following references: Ruczinski et al., 12 J. OF COMPUTATIONAL AND GRAPHICAL STATISTICS 475-511 (2003); Friedman, J. H., 84 J. OF THE AMERICAN STATISTICAL ASSOCIATION 165-75 (1989); Hastie, Trevor, Tibshirani, Robert, Friedman, Jerome, The Elements of Statistical Learning, Springer Series in Statistics (2001); Breiman, L., Friedman, J. H., Olshen, R. A., Stone, C. J. Classification and regression trees, California: Wadsworth (1984); Breiman, L., 45 MACHINE LEARNING 5-32 (2001); Pepe, M. S., The Statistical Evaluation of Medical Tests for Classification and Prediction, Oxford Statistical Science Series, 28 (2003); and Duda, R. O., Hart, P. E., Stork, D. G., Pattern Classification, Wiley Interscience, 2nd Edition (2001).

C. Determining Risk of Developing Aggressive Prostate Cancer

In a specific embodiment, the present invention provides methods for determining the risk or likelihood of having or developing aggressive prostate cancer in a patient. Biomarker percentages, ratios, amounts or patterns are characteristic of various risk states, e.g., high, medium or low. The risk of developing aggressive prostate cancer is determined by measuring the relevant biomarkers and then either submitting them to a classification algorithm or comparing them with a reference amount, i.e., a predefined level or pattern of biomarkers that is associated with the particular risk level.

D. Determining Aggressive Prostate Cancer Severity

In another embodiment, the present invention provides methods for determining the severity of aggressive prostate cancer in a patient. Each grade or stage of aggressive prostate cancer likely has a characteristic level of a biomarker or relative levels/ratios of a set of biomarkers (a pattern or ratio). The severity of aggressive prostate cancer is determined by measuring the relevant biomarkers and then either submitting them to a classification algorithm or comparing them with a reference amount, i.e., a predefined level or pattern of biomarkers that is associated with the particular stage.

E. Determining Aggressive Prostate Cancer Prognosis

In one embodiment, the present invention provides methods for determining the course of aggressive prostate cancer in a patient. Aggressive prostate cancer course refers to changes in aggressive prostate cancer status over time, including aggressive prostate cancer progression (worsening) and aggressive prostate cancer regression (improvement). Over time, the amount or relative amount (e.g., the pattern or ratio) of the biomarkers changes. For example, biomarker “X” may be increased with aggressive prostate cancer, while biomarker “Y” may be decreased with aggressive prostate cancer. Therefore, the trend of these biomarkers, either increased or decreased over time toward aggressive prostate cancer or non-aggressive prostate cancer indicates the course of the condition. Accordingly, this method involves measuring the level of one or more biomarkers in a patient at least two different time points, e.g., a first time and a second time, and comparing the change, if any. The course of aggressive prostate cancer is determined based on these comparisons.

F. Patient Management

In certain embodiments of the methods of qualifying aggressive prostate cancer status, the methods further comprise managing patient treatment based on the status. Such management includes the actions of the physician or clinician subsequent to determining aggressive prostate cancer status. For example, if a physician makes a diagnosis of aggressive prostate cancer, then a certain regime of monitoring would follow. An assessment of the course of aggressive prostate cancer using the methods of the present invention may then require a certain aggressive prostate cancer therapy regimen. Alternatively, a diagnosis of non-aggressive prostate cancer might be followed with further testing to determine a specific disease that the patient might be suffering from. Also, further tests may be called for if the diagnostic test gives an inconclusive result on aggressive prostate cancer status.

G. Determining Therapeutic Efficacy of Pharmaceutical Drug

In another embodiment, the present invention provides methods for determining the therapeutic efficacy of a pharmaceutical drug. These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern, profile or ratio) of one or more of the biomarkers of the present invention may change toward a non-aggressive prostate cancer profile. Therefore, one can follow the course of one or more biomarkers in the patient during the course of treatment. Accordingly, this method involves measuring one or more biomarkers in a patient receiving drug therapy, and correlating the biomarker levels/ratios with the aggressive prostate cancer status of the patient (e.g., by comparison to predefined levels/ratios of the biomarkers that correspond to different aggressive prostate cancer statuses). One embodiment of this method involves determining the levels/ratios of one or more biomarkers for at least two different time points during a course of drug therapy, e.g., a first time and a second time, and comparing the change in levels/ratios of the biomarkers, if any. For example, the levels/ratios of one or more biomarkers can be measured before and after drug administration or at two different time points during drug administration. The effect of therapy is determined based on these comparisons. If a treatment is effective, then the level/ratio of one or more biomarkers will trend toward normal, while if treatment is ineffective, the level/ratio of one or more biomarkers will trend toward aggressive prostate cancer indications.

H. Generation of Classification Algorithms for Qualifying Aggressive Prostate Cancer Status

In some embodiments, data that are generated using samples such as “known samples” can then be used to “train” a classification model. A “known sample” is a sample that has been pre-classified. The data that are used to form the classification model can be referred to as a “training data set.” The training data set that is used to form the classification model may comprise raw data or pre-processed data. Once trained, the classification model can recognize patterns in data generated using unknown samples. The classification model can then be used to classify the unknown samples into classes. This can be useful, for example, in predicting whether or not a particular biological sample is associated with a certain biological condition (e.g., diseased versus non-diseased).

Classification models can be formed using any suitable statistical classification or learning method that attempts to segregate bodies of data into classes based on objective parameters present in the data. Classification methods may be either supervised or unsupervised. Examples of supervised and unsupervised classification processes are described in Jain, “Statistical Pattern Recognition: A Review,” IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines).

Another supervised classification method is a recursive partitioning process. Recursive partitioning processes use recursive partitioning trees to classify data derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application Publication No. 2002/0193950 (Gavin et al. “Method or analyzing mass spectra”), U.S. Patent Application Publication No. 2003/0004402 (Hitt et al., “Process for discriminating between biological states based on hidden patterns from biological data”), and U.S. Patent Application Publication No. 2003/0055615 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows® or Linux™ based operating system. In embodiments utilizing a mass spectrometer, the digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including R, C, C++, visual basic, etc.

The learning algorithms described above are useful both for developing classification algorithms for the biomarkers already discovered, and for finding new biomarker biomarkers. The classification algorithms, in turn, form the base for diagnostic tests by providing diagnostic values (e.g., cut-off points) for biomarkers used singly or in combination.

### IV. KITS FOR THE DETECTION OF AGGRESSIVE PROSTATE CANCER BIOMARKERS

In another aspect, the present invention provides kits for qualifying aggressive prostate cancer status, which kits are used to detect the biomarkers described herein. In a specific embodiment, the kit is provided as an ELISA kit comprising antibodies and lectins to the biomarkers of the present invention including, but not limited to, PSA, UDP-glucuronosyltransferase, 2′-5′-oligoadenylate synthase-like protein, alpha-(1,6)-fucosyltransferase, annexin A1, G-protein coupled receptor 126, anterior gradient protein 2 homolog, calcium/calmodulin-dependent protein kinase II inhibitor 1, desmocollin-2 isoform Dsc2a preproprotein, interferon-induced protein with tetratricopeptide repeats 2, matrix metalloproteinase-15 preproprotein, interferon-induced protein with tetratricopeptide repeats 1, thymidylate synthase, interferon-induced protein with tetratricopeptide repeats 3, histone chaperone ASF1B, cyclin-dependent kinases regulatory subunit 2, nucleolar and spindle-associated protein 1, membrane associated phospholipase A2, ubiquitin-conjugating enzyme E2 C, solute carrier family 22 member 3, G2/mitotic-specific cyclin-B1, paternally-expressed gene 3 protein, PCNA-associated factor, paternally-expressed gene 3 protein, redox-regulatory protein FAM213A, kinesin family member 20A, cytosolic thymidine kinase, protein FAM83D, importin subunit alpha-1, transferrin receptor protein 1, hyaluronan mediated motility receptor, lymphokine-activated killer T-cell-originated protein kinase, transmembrane prostate androgen-induced protein, kallikrein-2 isoform 2 preproprotein, lipid phosphate phosphohydrolase 3, heparan-sulfate 6-O-sulfotransferase 1, prostatic acid phosphatase isoform PAP, nuclear RNA export factor 2, and protein POF1B, as well as post-translationally modified forms of the foregoing (glycosylation, fucosylation, etc.). Other biomarkers can include, but are not limited to, sTIE-2, sVEGFR-1, fucosylated TIMP-1, fucosylated DPP-4, and/or FUT8, and combinations of all of the foregoing.

The ELISA kit may comprise a solid support, such as a chip, microtiter plate (e.g., a 96-well plate), bead, or resin having biomarker capture reagents attached thereon. The kit may further comprise a means for detecting the biomarkers, such as antibodies, and a secondary antibody-signal complex such as horseradish peroxidase (HRP)-conjugated goat anti-rabbit IgG antibody and tetramethyl benzidine (TMB) as a substrate for HRP. In other embodiments, the kit can comprise magnetic beads conjugated to the antibodies (or separate containers thereof for later conjugation). The kit can further comprise detection antibodies, for example, biotinylated antibodies or lectins that can be detected using, for example, streptavidin labeled fluorescent markers such as phycoerythrin. The kit can be configured to perform the assay in a singleplex or multiplex format.

In a specific embodiment, a kit comprises (a) magnetic beads for conjugating to antibodies that specifically bind biomarker proteins of interest; (b) monoclonal antibodies that specifically bind the biomarker proteins of interest; (c) biotinylated immunoglobulin G detection antibodies; (d) biotinylated lectins that specifically bind the biomarker proteins of interest; and (e) streptavidin labeled fluorescent marker. In a more specific embodiment, the biomarkers proteins of interest comprises fucosylated PSA. In another embodiment, the kit is configured to detect fucosylatedPSA and one or more of biomarker proteins described herein. In certain embodiments, the kit is configured to detect fucosylated PSA and one or more (e.g., 1, 2, 3, 4, 5, 6 or 7) of transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B.

In certain embodiments, a patient can be diagnosed by adding blood or blood serum from the patient to the kit and detecting the relevant biomarkers conjugated with antibodies/lectins, specifically, by a method which comprises the steps of: (i) collecting blood or blood serum from the patient; (ii) separating blood serum from the patient's blood; (iii) adding the blood serum from patient to a diagnostic kit; and, (iv) detecting the biomarkers conjugated with antibodies/lectins. In this method, the antibodies/lectins are brought into contact with the patient's blood. If the biomarkers are present in the sample, the antibodies/lectins will bind to the sample, or a portion thereof. In other kit and diagnostic embodiments, blood or blood serum need not be collected from the patient (i.e., it is already collected). Moreover, in other embodiments, the sample may comprise a tissue sample or a clinical sample.

The kit can also comprise a washing solution or instructions for making a washing solution, in which the combination of the capture reagents and the washing solution allows capture of the biomarkers on the solid support for subsequent detection by, e.g., antibodies/lectins or mass spectrometry. In a further embodiment, a kit can comprise instructions for suitable operational parameters in the form of a label or separate insert. For example, the instructions may inform a consumer about how to collect the sample, how to wash the probe or the particular biomarkers to be detected, etc. In yet another embodiment, the kit can comprise one or more containers with biomarker samples, to be used as standard(s) for calibration.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Serum Fucosylated Glycoproteins Improve the Differentiation of Aggressive from Non-Aggressive Prostate Cancers

It is still challenging to separate aggressive from non-aggressive prostate cancers (Pca) by non-invasive approaches. Our recent studies showed that overexpression of alpha (1-6) fucosyltransferase played an important role in Pca cells. In this study, we have investigated levels of glycoproteins and their fucosylated glycoforms in sera of Pca patients, and the potential utility of fucosylated glycoproteins in the identification of aggressive Pca.

Briefly, serum samples from histomorphology-proven Pca cases were included. Prostate-specific antigen (PSA), tissue inhibitor of metallopeptidase 1 (TIMP1) and tissue plasminogen activator (tPA), and their fucosylated glycoforms were captured by Aleuria Aurantia Lectin (AAL), followed by multiplex magnetic bead-based immunoassay. The level of fucosylated glycoproteins was correlated with patients' Gleason score of the tumor.

Among three fucosylated glycoproteins, the fucosylated PSA was significantly increased and correlated with the tumor Gleason score (p<0.05). The ratio of fucosylated PSA showed a marked increase in aggressive tumors in comparison to non-aggressive tumors. ROC analysis also showed an improved predictive power of fucosylated PSA in the identification of aggressive Pca.

Accordingly, the data demonstrate that fucosylated PSA has a better predictive power to separate aggressive tumors from non-aggressive tumors, than that of native PSA and the other two glycoproteins. The fucosylated PSA has the potential to be used as a surrogate biomarker.

### Introduction

Glycosylation is one of the most common post-translation modifications of proteins and play important roles in cellular functions and cancer biology. Studies have shown that aberrant glycosylations occur in many intracellular signaling pathways and eventually lead to the development of cancers. Currently, most clinical cancer biomarkers are glycoproteins, such as PSA for Pca, alpha-fetoprotein (a-AFP) for hepatocellular carcinoma (HCC), and carbohydrate antigen 125 (CA125) for ovarian cancer. In addition to cancer-specific glycoproteins, it has also been suggested that specific glycoforms of glycoproteins may be involved in a particular disease and/or subtype of cancers. For example, AFP-L3 is a core-fucosylated glycoform of AFP detected in serum of HCC patients, and it provides an improved specificity for diagnosing HCC. Aberrant glycosylation of glycoproteins has also been related to the accelerated tumor growth and the development of metastasis in a variety of cancers, features seeing in aggressive cancers. Taken together, these findings indicate that the specific glycoform of glycoproteins have the potential to be used as biomarkers not only to improve the diagnostic accuracy of cancer, but also to separate AG tumors.

In this study, we analyzed serum samples from Pca patients using multiplex immunoassay, based on lectin-affinity capturing of fucosylated glycoprotein and protein-antibody immunoreactivity. Levels of glycoproteins and their fucosylated glycoforms were detected and correlated with the Gleason score of the tumor. The purposes of our study are to identify fucosylated glycoproteins in serum samples from Pca patients, and to evaluate their potential clinical utility in the separation of AG from NAG tumors.

### Materials and Methods

Serum Sample Collection.

Serum samples from 47 Pca patients were collected from the Johns Hopkins hospitals. All patients had either biopsy or surgical resection of the tumor. The criteria of International Society of Urological Pathology (ISUP) Consensus were used to determine Gleason scores of tumors. Serum samples were aliquoted and stored at −80° C. prior to the analysis. Each serum sample underwent no more than three freeze/thaw cycles prior to the test. The clinical information, including serum PSA levels and the Gleason score of the tumor were correlated. The use of clinical samples was approved by the Johns Hopkins Institutional Review Board. All study cases were annotated with available clinical information in a manner that protected patient identity.

Reagents.

Agarose-bound Aleuria Aurantia Lectin (AAL) beads were purchased from Vector Labs (Burlingame, Calif.). Multiscreen filter plates were from Millipore (Billerica, Mass.). Bio-Plex Pro™ magnetic COOH beads, amine coupling kits, and cytokine assay kits were purchased from Bio-Rad Laboratories (Hercules, Calif.). Biotinylated AAL was purchased from Vector Labs (Burlingame, Calif.). Biotinylated detection antibody was prepared with Thermo Scientific (Rockford, Ill.) EZ-link Sulfo-NHS-Biotin (Catalog #21326).

Human recombinant PSA (Catalog #PO725), human PSA mouse monoclonal antibody (Catalog #MP077-BP001) for capture, and biotinylated mouse monoclonal antibody (Catalog #MP007-AP002S) for detection were purchased from Scripps Laboratories (San Diego, Calif.). Mouse myeloma cell line NS0-derived human recombinant TIMP1 (Catalog #970-TM-010), human TIMP1 mouse monoclonal IgG2B antibody (Catalog #MAB970, clone #63515) for capture, and biotinylated human TIMP1 goat polyclonal IgG antibody (Catalog # BAF970) for detection were purchased from R&D Systems (Minneapolis, Minn.). Chinese Hamster ovary cell line CHO-derived human recombinant tPA protein (Catalog #ab92637), human tPA mouse monoclonal antibody (Catalog # ab82249) for capture, and biotinylated human tPA rabbit polyclonal IgG antibody (Catalog #ab28208) for detection were purchased from Abcam (Cambridge, Mass.).

Capture of Fucosylated Glycoproteins.

Agarose AAL Lectin beads were deposited 100 μl per well into the multiscreen filter plates and subsequently washed three times with 150 μl of sample diluent (from the Cytokine Assay Kit) as the binding buffer via centrifugation. The Multiscreen filter plates containing the agarose beads were then mixed well with sample diluent on a shaker for 10 minutes and centrifuge at 2700 rpm for 5 minutes to remove the solution. Thirty microliter of serum sample was diluted with sample diluent at 1:4 ratios to a total volume of 120 μl. Then, the diluted sera at 120 μl per well were added to multiscreen filter plates containing agarose beads and incubated on a shaker for 1 hour at room temperature. After incubation, flow through was collected by centrifuging at 2700 rmp for 5 min. Then, AAL beads were washed three times with sample diluent to remove non-specific bindings. Target glycoproteins were eluted out with 120 μl of 100 mM fucose in sample diluent by gentle shaking on a shaker for lhr and elution was collected by centrifugation.

Detection of Glycoproteins.

Following manufacturer's protocol, capture antibodies of PSA, TIMP1 and tPA were coupled to Bio-Plex Pro™ magnetic COOH beads using the BioRad Amine Coupling Kit. The magnetic beads were validated with IgG antibodies and determined its beads concentration with hemocytometer before storage at 4° C.

Fifty microliter of the serum samples obtained using the AAL glycoprotein capturing method stated above were incubated with 2500 coupled magnetic beads per antibody for 1 hour at the room temperature. Prior to perform multiplex assay, biotinylated detection antibody of PSA, TIMP1 and tPA were prepared and diluted to 2 μg/mL with detection antibody diluent (supplied in the Cytokine Assay kit). After incubation of samples with magnetic beads, the beads were washed and incubated with 25 μl of detection antibody mixture for 30 minutes at the room temperature. Once again, the beads were washed before incubation with 50 μL of 2 μg/mL streptavidin-phycoerythrin for 10 minutes at the room temperature. After washing steps, the individual glycoprotein was analyzed by the multiplex assays using the Bioplex 200 System.

For the multiplex immunoassay, three calibration curves were established using 8 calibrators of 100, 25, 6.25, 1.56, 0.39, 0.1, 0.025, and 0 ng/mL of human recombinant PSA, tPA or TIMP1. The same calibrators were used in for comparisons of the multiplex and single immunoassys. Calibration curves for protein quantification were established using the 5-parameter nonlinear regression model in Bio-Plex Manager™ 6.0. Protein concentrations were calculated using the calibration curves and reported by Bio-Plex Manager™ 6.0.

Data Analysis.

Statistical analysis and linear regression were performed by the KaleidaGraph (version 4.5.0, Synergy Software). The predictive power of individual glycoprotein was assessed using the receiver operating characteristics (ROC) curve; and the area under curve (AUC) value was calculated as an indication of prediction accuracy in the validation. The ROC curves were generated using the program written in Matlab. Kolmogorov-Smirnove test (K-S test) was used to compare the result of two ROC analyses. A P-value of <0.05 was considered as statistically significant.

### Results

Clinical Information.

A total of 47 histomorphology-proven Pca patients were included in our study. The average age of patients was 60.0±7.9 years (ranged from 44 to 79 years). The average level of serum PSA was 15.13±2.14 ng/mL, ranging from 1.9 to 54.5 ng/mL. Among patients, 29.8% tumors (n=14) were Gleason score 6, 27.7% (n=13) were Gleason score 7, 21.3% (n=10) were Gleason score 8, and 21.3% (n=10) were Gleason score 9. The Gleason scores of 47 tumors at the initial diagnosis and patients' corresponding serum PSA levels were summarized in Table 1. The average levels of serum PSA in the Gleason score 6, 7, 8 and 9 are 9.3±2.1, 6.1±1.5, 19.9±4.7 and 30.2±5.5 ng/mL. Our data also demonstrated that serum PSA was not always elevated in high Gleason score tumors as indicated in Table 1. For example, the serum PSA level in the Gleason score 6 tumor ranged from 1.9 to 26.6 ng/mL, whereas, the average serum PSA level in the Gleason score 9 tumor ranged from 2.2 to 54.5 ng/mL.

TABLE 1
Correlation of Gleason scores of tumors with patients' age and serum PSA levels.

| | | | Patients Age | Serum PSA Level |
|---|---|---|---|---|
| | Gleason Score of the tumor | | Average ± SD (range) | X ± SE (range) |
| Cases | Number (%) | | years | ng/mL |
| Pca | Gleason Score 6 | 14 (29.8%) | 59.4 ± 6.8 (49-72) | 9.3 ± 2.1 (1.9-26.6) |
| (n = 47) | Gleason Score 7 | 13 (27.7%) | 60.1 ± 5.9 (50-74) | 6.1 ± 1.5 (2.1-21.1) |
| | Gleason Score 8 | 10 (21.3%) | 58.8 ± 7.6 (44-79) | 19.9 ± 4.7 (3.2-49.7) |
| | Gleason Score 9 | 10 (21.3%) | 62.7 ± 8.12 (49-75) | 30.2 ± 5.5 (2.2-54.5) |

Pca: prostate cancer.
PSA: prostate-specific antigen.

### Serum Glycoproteins and their Fucosylated Forms in Pca Patients

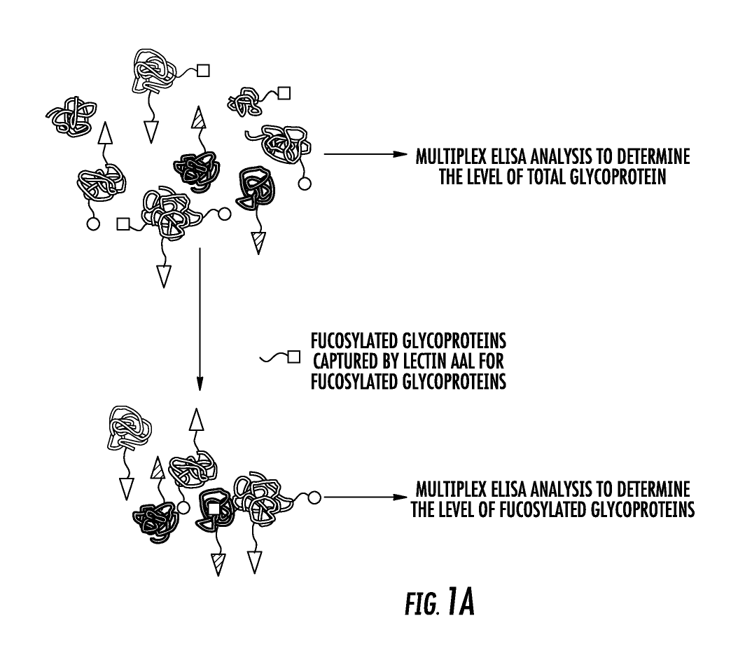

Glycoproteins in sera of Pca patients were analyzed using our recently developed multiplex immunoassay with modifications. See Li et al., 59(1) CLIN. CHEM. 315324 (2013). The system contains two steps, lectin AAL affinity capture and monoclonal antibody detection, based on protein sequences and glycan structure/linkage (e.g., core α1-6- and α1-3-linked fucosylation). Briefly, AAL lectin beads were used to capture glycoproteins containing fucosylated glycans, then, individual glycoproteins was identified by protein-antibody immunoassay (FIG. 1A). The standard curves of individual glycoproteins were established using human recombinant PSA, TIMP1 and tPA, and were used for quantifications of serum glycoproteins (FIG. 1B). By using this approach, we were able to detect not only candidate glycoproteins in serum but also their fucosylated forms.

The levels of glycoprotein PSA, TIMP1 and tPA, and their fucosylated forms, were summarized in the Table 2. The average serum levels of PSA, TIMP1 and tPA was 15.13±2.14 ng/mL, 80.80±4.44 ng/mL and 4.89±0.32 ng/mL, whereas, the average levels of fucosylated PSA, TIMP1 and tPA were 6.27±1.99 ng/mL, 25.34±1.84 ng/mL and 1.59±0.13 ng/mL.

TABLE 2
Serum levels of glycoproteins and their fucosylated glycoforms
in prostate cancer patients detected by multiplex immunoassay.

| | Glycoproteins | Fucosylated form | |
|---|---|---|---|
| | X ± SD (range) | X ± SD (range) | % Fucosylated |
| | ng/mL | ng/mL | X ± SD (range) |
| PSA | 15.13 ± 2.14 | 6.27 ± 1.99 | 27.01% ± 3.62% |
| | (1.93-54.47) | (0.24-8.76) | (8.31%-170.06%) |
| TIMP1 | 80.80 ± 4.44 | 25.34 ± 1.84 | 34.42% ± 2.83% |
| | (40.75-173.41) | (8.28-58.36) | (8.66%-92.87%) |
| tPA | 4.89 ± 0.32 | 1.59 ± 0.13 | 33.95% ± 1.94% |
| | (1.12-10.39) | (0.47-5.82) | (18.31%-80.06%) |

PSA: prostatic-specific antigen;
TIMP1: tissue inhibitor of metallopeptidase 1;
tPA: tissue plasminogen activator;
Pca: prostate carcinoma.

Correlation of Serum Glycoproteins with Tumor Gleason Scores.

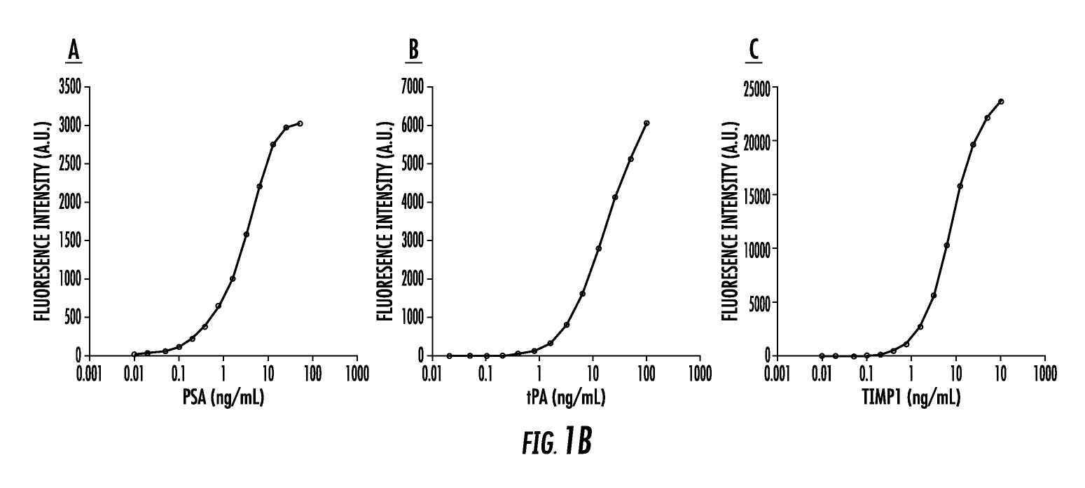

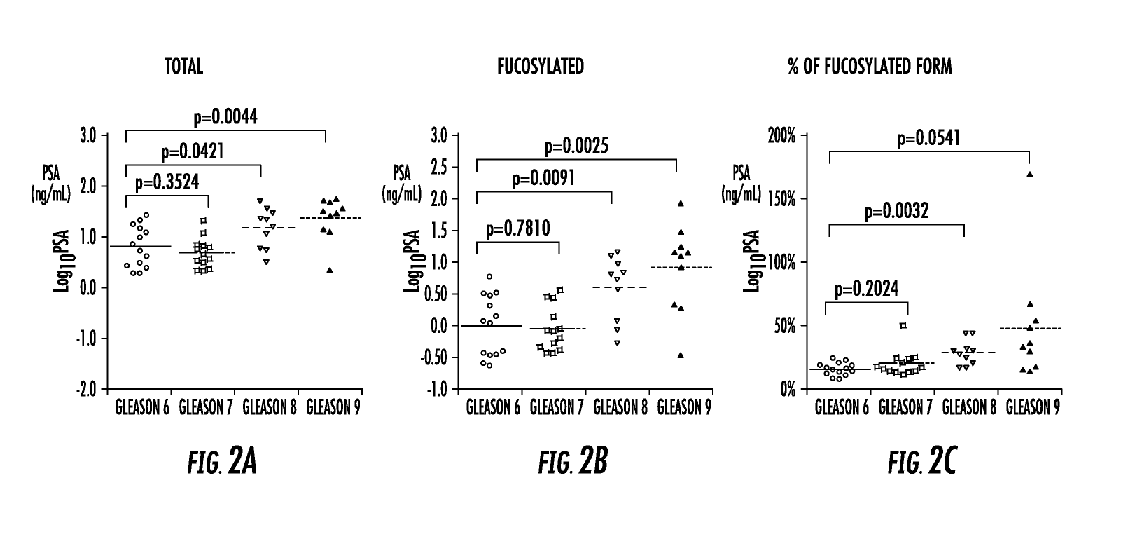

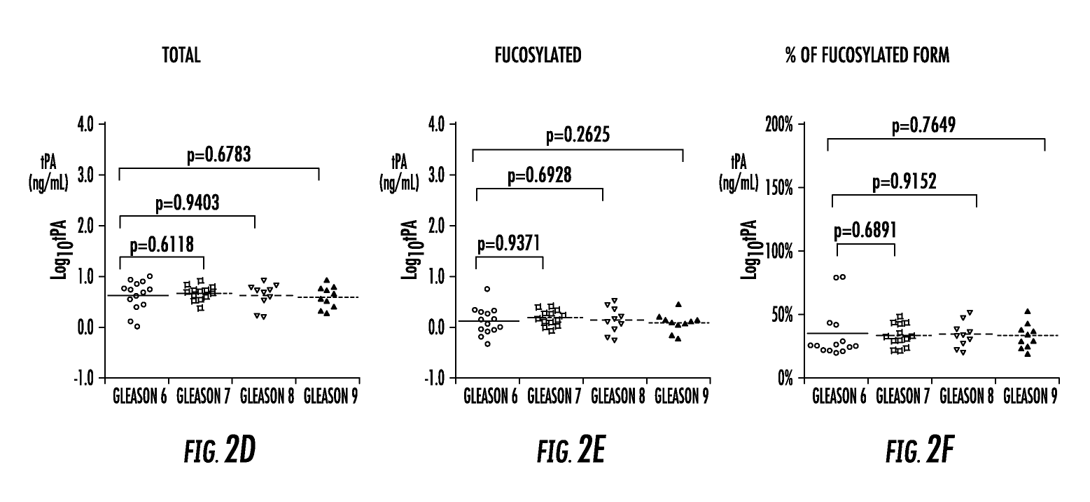

Three serum glycoproteins of PSA, TIMP1 and tPA showed variable levels in Pca patient sera when correlated with patients' tumor Gleason scores (FIG. 2). Of PSA, both total and fucosylated PSA were correlated with the Gleason score of tumors, particularly in tumors with the Gleason score >6 in the comparison with tumors whose Gleason score was equal to 6 with p values of 0.1049 and 0.0146 respectively (Table 3 and FIG. 3). The difference was more obvious when the ratio of fucosylated PSA (fucosylated PSA/total PSA) was used (p=0.0053, Table 3 and FIG. 3). The correlation coefficient of total serum PSA versus fucosylated PSA in patients with different Gleason scores was summarized in FIG. 4. The levels of total PSA and fucosylated PSA showed strong correlation coefficients in groups of Gleason score 6 and Gleason score >6 tumors. The slopes of the linear equation in Gleason 6 and >6 group are quite different, 0.208 and 0.692, respectively.

TABLE 3
Correlation of serum glycoproteins and their fucosylated glycoforms with tumor
Gleason scores in prostate cancer patients.

| | Serum glycoproteins | | | Fucosylated form | | | % of Fucosylated form | | |
|---|---|---|---|---|---|---|---|---|---|
| | X ± SE, ng/mL | | | X ± SE, ng/mL | | | X ± SE | | |
| | Gleason 6 | Gleason 7-9 | P value | Gleason 6 | Gleason 7-9 | P value | Gleason 6 | Gleason 7-9 | P value |
| PSA | 9.32 ± 2.14 | 17.59 ± 2.82 | 0.1049 | 1.69 ± 10.46 | 8.21 ± 2.76 | 0.0146 | 16.3 ± 1.3 | 34.9 ± 5.4 | 0.0053 |
| TIMP1 | 73.56 ± 6.33 | 83.88 ± 5.70 | 0.3122 | 26.73 ± 2.09 | 24.76 ± 2.48 | 0.1777 | 39.2 ± 4.4 | 32.4 ± 3.6 | 0.2357 |
| tPA | 5.20 ± 0.76 | 4.67 ± 0.33 | 0.9812 | 1.66 ± 0.35 | 1.55 ± 0.12 | 0.8275 | 34.9 ± 5.4 | 33.6 ± 1.6 | 0.8552 |

PSA: prostatic-specific antigen;
TIMP1: tissue inhibitor of metallopeptidase 1;
tPA; tissue plasminogen activator;
Pca: prostate cancer.

Of TIMP1, the total levels of the protein were not significantly changed among tumors with different Gleason scores (Table 3, FIG. 2 and FIG. 3). Although the fucosylated forms showed a decreased trend in patients with Gleason score >6 tumors, it did not reach the statistical significance (P>0.05) (Table 3). Similar to TIMP1, tPA did not show significant changes of either total or fucosylated form between Gleason score >6 and Gleason 6 the tumor Gleason scores (P>0.05) (Table 3, FIG. 2 and Table 3).

Taken together, our data demonstrated that PSA, including its native and fucosylated forms, was clearly superior to that of TIMP1 and tPA in the separation Gleason scores >6 from 6 tumors, and these changes were correlated significantly with tumor Gleason scores (FIG. 2, Table 3). It is also interesting that the ratio of fucosylated PSA was significantly elevated in Pca patients.

Fucosylated PSA in the Separation of Gleason Score >6 from Gleason Score 6 Tumors.

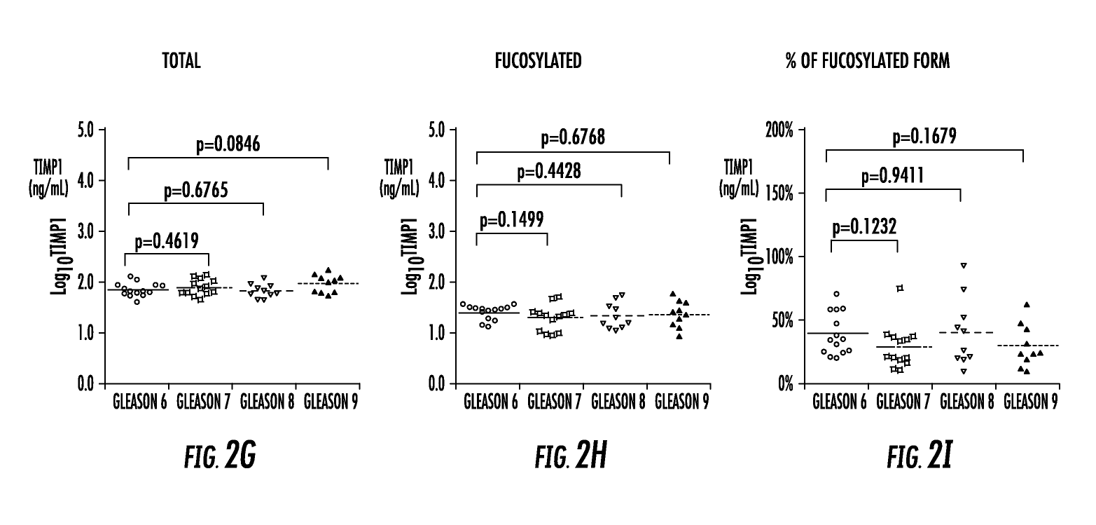

We analyzed serum PSA, fucosylated PSA, and the ratio of fucosylated PSA (fucosylated PSA/total PSA) in the separation of Gleason score >6 from 6 tumors by receive operating characteristic (ROC) (FIG. 5). We also compared the performance of PSA with TIMP1, since the fucosylated TIMP1 showed variable changes in Pca.

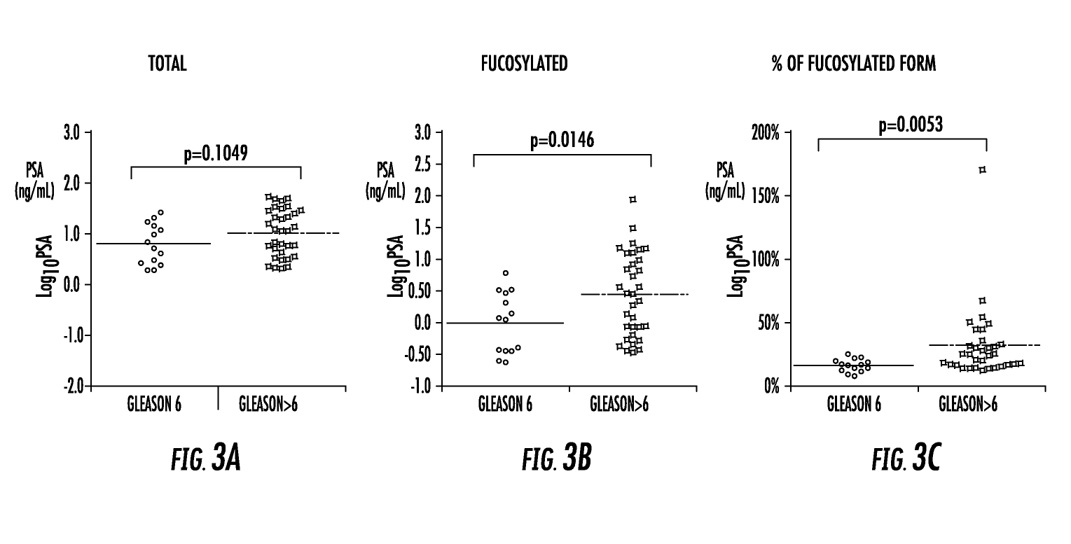

Between Gleason score 6 and Gleason score >6 tumors, the fucosylated PSA achieved a better predictive power (AUC=0.7056) when compared with the total serum PSA (AUC=0.6558). Moreover, by using the ratio of fucosylated PSA as predictive marker, it achieved even more significantly better performance when compared with the total serum PSA (AUC=0.7762, P<0.05, P=0.036). We further investigated the performance of fucosylated PSA and the ratio of fucosylated PSA in the separation of Gleason score 8-9 tumor from 6 tumors by ROC (FIG. 6). They showed higher predictive powers (AUC=0.8393 and 0.8643, respectively). Our data demonstrated that the fucosylated PSA, particularly the ratio of fucosylated PSA, can significantly improve the predictive power and might provide additional information in separating Gleason score >6 from Gleason score 6 tumors.

In comparison to PSA, native TIPM1 had a suboptimal performance (AUC=0.4416). Similar, both fucosylated TIMP1 and the ratio of fucosylated TIMP1 showed improved predictive powers (AUC=0.6234 and 0.6515, respectively). However, the overall performance of fucosylated TIMP1 was still suboptimal than that of fucosylated PSA (FIG. 5 and FIG. 6).

### Discussion

The recent U.S. Prostate, Lung, Colorectal, and Ovarian Cancer Screening Trial (PLCO) has shown that among clinically diagnosed Pca, 77.2% of them are Gleason score 6 tumors and 22.8% are Gleason score great than 6 tumors. Pca with the Gleason score 6 or less, and been considered a clinically indolent/non-aggressive tumor, which is unlikely to cause significant symptoms or mortality. Pca with Gleason score greater than 6, particularly those with Gleason score great than 7 tumors, have been considered to be AG tumors that need an optimal clinical management. The evaluation of Gleason score of the tumor is performed by an invasive biopsy procedure, which has the risk of developing serious complications. Clinically, it is notoriously difficult to separate aggressive tumors from indolent low Gleason score non-aggressive tumors without invasively biopsying of the tumor tissues. Although a high level of serum PSA has been considered an indicator of a clinical aggressive tumor, our study and others have shown that not all patients with aggressive tumor has elevated levels of serum PSA. Furthermore, the U.S. Preventive Services Task Force (USPSTF) has recently recommended against PSA-based screening for Pca (D recommendation) due to overdiagnosis (approximately 80% of PSA test results are false-positive when cutoffs between 2.5 to 4.0 ug/L are used), and lack of evidence to improve Pca patient survival. Thus, it is crucial to separate aggressive tumors from non-aggressive tumors using a non-invasive approach to guide the optimal management of the Pca patient.

In this study, we analyze serum levels of PSA, TIMP1 and tPA, and their fucosylated glycoforms in Pca patients using the multiplex immunoassay, and correlate levels of these serum glycoproteins with the Gleason score of the tumor. All Pca patients in our study have either biopsy proven or surgical resection of the tumor, which confirms the histological diagnosis and the Gleason score of the tumor. By analyzing total serum PSA, TIMP1, and tPA, and their AAL-bound fucosylated glycoproteins in well-annotated prostate cancer patients, we demonstrate that levels of fucosylated PSA, TIMP1, tPA are differentially present. Among these fucosylated glycoproteins, changes of TIMP1 and tPA as well as their fucosylated forms are not significantly different between Gleason score <6 and >6 tumors (P>0.05). Only fucosylated PSA is significantly elevated and positively correlated with the tumor Gleason scores. The fucosylated PSA is 1.69±0.46 ng/mL in patients with Gleason score 6 tumor, and 8.21±2.76 ng/mL in patients with Gleason score >6 tumor (p<0.05).

Based on above findings, we further analyze the ratio of fucosylated PSA in total serum PSA, and find that the ratio of fucosylated PSA is 16.3%±1.3% in Gleason score 6 tumors and 34.9%±4.9% in Gleason score >6 tumors, respectively (P<0.01). ROC analyses have shown that the fucosylated PSA has achieved a better predictive power for identification of AG tumors (Gleason score >6 tumors) when compared with the total serum PSA as well as other fucosylated glycoproteins. Moreover, by using the ratio of fucosylated PSA as predictive marker, it achieved even more significantly better performance for the identification of AG tumors, representing in Gleason score of 8-9 group. Our findings indicate that the measurements of fucosylated PSA may provide valuable clinical information to aid in the separation of AG from NAG tumor without biopsying the tumor. The detection of fucosylated PSA in serum is a minimally invasive procedure, therefore, it could be used as a surrogate test to separate AG/high Gleason score tumors from NAG tumors and to guide optimal clinical management for Pca patients.

In Pca, several recent studies using mass spectrometry (MS)-based proteomics have found that glycoproteins, including tissue inhibitor of metallopeptidase 1 (TIMP1), tissue plasminogen activator (tPA) and membrane metallo-endopeptidase (MME), and dipeptidyl peptidase-IV (DPP-4) are differentially expressed in Pca tumor tissue. Recently, we have developed a novel multiplex detection system to further analyze these glycoproteins and their glycoforms in tumor tissues as potential candidate biomarkers for the separation of AG from NAG; we have found that several aberrant glycosylations of these proteins are present in tumor tissues. For example, increased β1-6 branching of N-glycans and α1-2 fucosylation have been detected by phytohemagglutinin-L (PHA-L) and ulex europaeus agglutinin (UEA) lectin affinity chromatography. Others using quantitative real-time polymerase chain reaction (RT-PCR) analysis of glycosyltransferases in Pca cell lines and tumor tissue have demonstrated elevated mRNA levels of fucosyltransferase 1 (Fut1) genes in cancer cases. Saldova et al. have reported that levels of core-fucosylated bianternnary glycans are significantly increased in serum from Pca patients. Several recent studies have also demonstrated the aberrant fucosylation in Pca. Kyselova et al. describes a significant increase in fucosylation in 24 Pca patients' sera in comparison to 10 healthy control males, and the elevated fucosylation has been found in metastatic Pca. Our recent study demonstrates that the overexpression of fucosyltransferase (FUT8) in prostate cancer tissues, the major enzyme responsible for alpha (1,6) core fucosylation, is correlated with high Gleason scores of the tumors, and is detected in metastatic Pca. These studies demonstrate that fucosylation plays an important role in Pca, and fucosylated PSA from serum could be more specific to the aggressiveness of prostate cancer. For the first time, we demonstrated that fucosylated serum PSA can be used to distinguish the aggressive prostate cancer from non-aggressive cancer.

In summary, we used a non-invasive approach and analyzed serum fucosylated glycoproteins from Pca patients using multiplex immunoassay, based on AAL lectin-affinity capturing and protein-antibody immunoreactivity. Our data demonstrate that the fucosylated PSA is elevated and correlated with tumor Gleason scores. The fucosylated and the ratio of fucosylated PSA have better predictive powers to separate Gleason score >6 tumors representing aggressive Pca, than that of native PSA and other fucosylated glycoproteins. Our data suggests that the fucosylated PSA has the potential to be used as a biomarker to separate aggressive from non-aggressive prostate cancers.

### Example 2

### Integrated Proteomic and Glycoproteomic Analyses of Prostate Cancer Cells Reveals Glycoprotein Changes in Protein Expression, Glycosylation Occupancy and Glycosite Heterogeneity

Prostate cancer weighs heavily on the U.S. and other world populations and androgen-deprivation therapy (ADT) remains the principal treatment for patients. Although a majority of patients initially respond to ADT, most will eventually develop castrate resistance. An increased understanding of the mechanisms that underlie the pathogenesis of castrate resistance and identify the cell surface or secreted proteins is therefore needed to develop novel therapeutic approaches and/or to develop diagnostic tests for castrate resistance disease. LNCap and PC3 are prostate cancer cell lines that have been used as cell models for androgen-dependent cancer cell line and androgen-independent cell line. Herein, we report the analysis between these two prostate cancer cell lines using integrated proteomics and glycoproteomics. Global proteomes of the cell lines using iTRAQ labeling and 2D liquid chromatography tandem mass spectrometry led the quantification of 8063 proteins with 637 protein increase and 410 protein decrease. Changes in enzymes of glycan biosynthesis pathways such as (1,6) fucosyltransferase (FUT8) were also observed indicating potential differences of glycan structures in the two cell lines. To determine which of the altered proteins were glycoproteins that could be used as cell surface therapeutic targets or secreted diagnostic proteins, glycosite-containing peptides were isolated from the two cell lines using solid phase extraction followed by liquid chromatography and tandem mass spectrometry analysis. Among the 1810 unique N-linked glycosylation site-containing peptides from 653 N-glycoproteins identified, 176 glycoproteins were observed to be differentially expressed between the two cell lines. A majority of the differentially expressed glycoproteins were also observed in global protein expression changes. However, 23 differentially expressed glycoproteins showed no change at the protein levels indicating glycosylation site occupancy was different between the two cell lines. To determine the glycosylation heterogeneity at specific glycosylation sites, we further identified and quantified 1145 N-linked glycopeptides with attached glycans. These intact glycopeptides accommodated 67 glycan compositions and showed increased fucosylation in a number of glycosylation sites examined To determine the function of fucosylation in prostate cancer cells, we compared the protein profiles of FUT8 overexpressed LNCap cells and the parental cells. Among the 7217 protein quantified, we observed 31 proteins increased and 8 proteins decreased in FUT8 overexpressed cells. Our data showed that N-glycoproteins with changes in protein, in glycosylation occupancy, or glycosylation levels were regulated by FUT8. The altered protein fucosylation forms have great potential in aiding our understanding of castrate resistance and may lead to the novel therapeutic approaches and specific detection for this disease.

### Introduction

Androgen is important for the development, function and proliferation of both normal and cancerous prostate cells. At the earliest stage of prostate cancer, prostate cancer cells are dependent on the presence of androgen, and androgen-deprivation therapy (ADT) is used to treat prostate cancer. However, androgen-independent cells rise from androgen deprivation therapy and become more aggressive cancer cells. This results in androgen independent remission of the disease. The LNCap and PC3 cell lines have been widely used as models of prostate cancer cells. LNCap is an androgen dependent cancer cell line, whereas PC3 cell line is an androgen independent cell line. The LNCap cell line is less aggressive as compared to PC3, which has a high metastatic potential. LNCap and PC3 have been previously studied by genomics and proteomics approaches to understand the mechanism(s) responsible for the occasional aggressive and metastatic nature of prostate cancer. Post-translational modifications (PTMs) such as phosphorylation are important in the function of the androgen dependent pathway. Androgen receptors bind to androgen and are then phosphorylated before translocating into the nucleus. However protein PTMs cannot be directly informed from gene expression. Regulation of PTMs is not clear, in part due to a lack of understanding of the underlying mechanisms.

Glycosylation is an abundant PTM and most of cell surface or secreted proteins are expected to be glycosylated. Glycosylation is one of the more complex phenomena due to the fact that different glycosylation machineries present in different cells, multiple glycosylation sites exist on many glycoproteins and each glycosylation site can be modified by a large number of different glycans. Such microheterogeneity of glycan structures at each glycosylation site with different site occupancy significantly increases structural diversity of each glycoprotein that is specific to the microenvironment of cells where each glycoprotein is produced. While these characteristics of protein glycosylation pose considerable challenges to the structural and functional analyses of glycoproteins, we expect that cell and cell microenvironment-specific glycoproteins are present to different cells and their physiological and pathological states. Aberrant glycosylation is the result of alterations in glycosylation genes that may lead to cancer development. A systematic approach to analyze proteins, glycoproteins, and glycosylation is expected to identify the specific glycoproteins to each cell state and help to understand the functions of glycosylation since alterations in glycosylation can modulate the conformation of a protein, thus affecting glycoprotein function. A close association between the alterations in glycoprotein structures in cancer cells with different behavior is needed in both understanding tumor biology and in functioning as a therapeutic target or diagnostic biomarker.

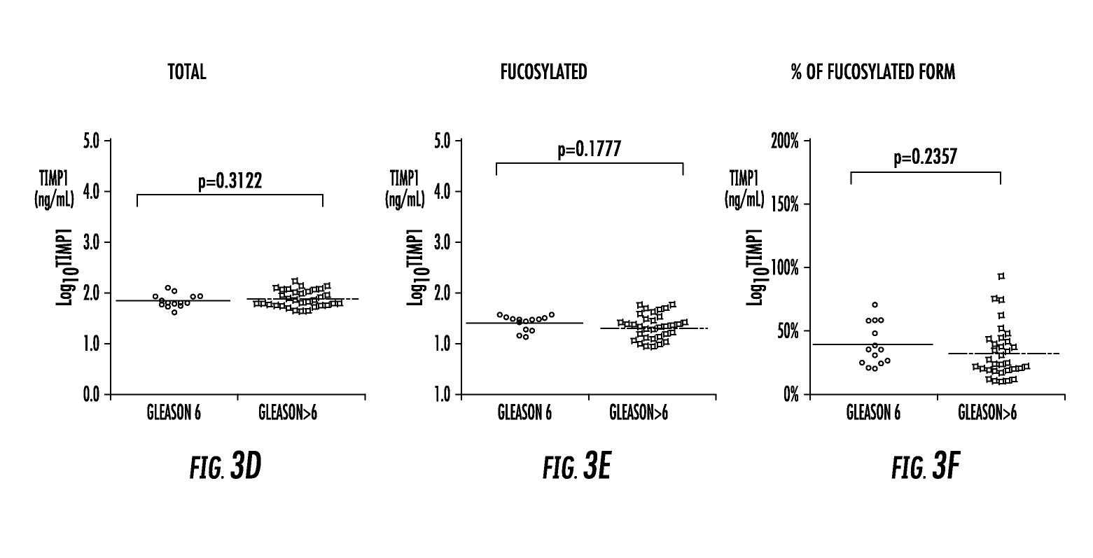

In this study, a comprehensive proteomic and glycoproteomic study was designed to investigate the differential expression of proteins and glycoproteins between LNCap and PC3 cells to identify changes in protein expression, glycosylation occupancy and site-specific glycosylation forms to understand prostate cancer cells (FIG. 7).

### Materials and Methods

Cell Lines and Culture Conditions.

Human prostate cancer cell lines were described in our previous publication. See Wang et al., 24(10) GLYCOBIOL. 935-44 (2014). The cells were used for proteomic analysis at 80-90% confluence. The cells were washed six times with ice-cold phosphate buffered saline prior to cell lysis for protein extraction.

Protein and Peptide Extraction from Cells for Proteomic Analysis.

For protein digestion, the cell pellet from two 10 cm dishes was first denatured in 1 ml of 8M urea and 0.4M NH₄HCO₃ and sonicated thoroughly. The protein concentration was measured using a BCA protein assay kit (Thermo) with at least a 3-fold dilution of protein. The proteins were then reduced by incubating in 120 mM Tris (2-carboxyethyl)phosphine for 30 min and alkylated by addition of 160 mM iodoacetamide at room temperature for 30 min in the dark. Sample was diluted with buffer (100 mM Tris-HCl, pH 7.5) containing 0.5 ug/ul trypsin and incubated at 37° C. overnight. The digested proteins were checked for completion of trypsin digestion using SDS-PAGE and silver staining. Peptides were purified with C18 desalting columns and dried using SpeedVac.

iTRAQ Labeling of Global Tryptic Peptides from Cell Lines.

Each iTRAQ (isobaric tags for relative and absolute quantitation) 4-plex reagent was dissolved in 70 μl of methanol. 1 mg of each tryptic peptide sample was added into 250 μl of iTRAQ dissolution buffer, then mixed with iTRAQ 4-plex reagent and incubated for one hour at room temperature. iTRAQ channels 114 and 115 were used to label two replicate LNCap samples in order to determine the analytical reproducibility, iTRAQ channel 116 was used to label peptides from PC3 cells and iTRAQ channel 117 was used for labeling peptides from another cell line unrelated to this study. After iTRAQ labeling, the 4 sets of tagged peptides were combined and purified by SCX column. Then, 5% of the labeled peptides were dried and resuspended into 0.4% acetic acid solution prior to fractionation for mass spectrometry analysis. The remaining peptides were desalted for glycopeptide capture.

Glycopeptide Capture.

Glycosite-containing peptides were extracted from tryptic peptides using solid-phase extraction of glycosite-containing peptides (SPEG). Zhang et al., 21 NATURE BIOTECHNOLOGY 660-66 (2003). Briefly, 90% of the iTRAQ labeled tryptic peptides (3.6 mg) were dissolved in 5% ACN in 0.1% TFA followed by addition of 1/10 of the final volume of 100 mM sodium periodate to samples. The samples were incubated with hydrazide beads for one hour at room temperature in the dark with gentle shaking. Beads were then washed to remove any non-specific binding. PNGaseF was used to detach glycosite-containing peptides from glycans conjugated on the beads.

Chromatography Fractionation.

The global peptide mixture resulting from 100 ug of iTRAQ-labelled tryptic peptides was fractionated into 24 fractions by reverse phase liquid chromatography (bRPLC) on a 1200 Infinity LC system (Agilent Technology, Santa Clara, Calif.) utilizing a 4.6×100 mm BEH120 C-18 column (Waters, Milford, Mass.). Samples were adjusted to a basic pH using 1% ammonium hydroxide. For bRPLC Solvent A was 7 mM tri-ethyl ammonium bicarbonate (TEAB), while Solvent B was 7 mM TEAB in 90% acetonitrile. A total of 96 fractions were collected and concatenated into 24 fractions. The glycosite-containing peptides isolated by SPEG were not further fractionated but analysed directly by LC-MS/MS.

LC-MS/MS Analysis.

The peptide samples were separated utilizing a Dionex Ultimate 3000 RSLC nano system (Thermo Scientific) with a 75 μm×50 cm C18 PepMap RSLC column (Thermo Scientific) protected by a 5 mm guard column (Thermo Scientific). Flow rate was 0.350 μl/min with 0.1% formic acid and 2% acetonitrile in water (A) and 0.1% formic acid 95% acetonitrile (B). The peptides were separated with a 5-40% B gradient in 104 mins, MS analysis was performed using a Thermo Q Exactive mass spectrometer (Thermo Scientific). AGC target for MS1 was set for 3×10⁶ for MS1 in 60 ms maximum time. AGC target for MS/MS was 50000 (at a resolution of 17,500, High-energy collisional dissociation (HCD) and maximum IT 100 ms) of the 20 most abundant ions. Charge state screening was enabled to reject unassigned, one, eight and more than eight protonated ions. A dynamic exclusion time of 25 sec was used to discriminate against previously selected ions.

Data Analysis.

Data generated was searched using SEQUEST in Proteome Discoverer 1.3 (Thermo Scientific, Rockford, Ill.) against the Human RefSeq database. Peptides were searched with two tryptic ends, allowing only two missed cleavages. Search parameters used were 10 ppm precursor tolerance for precursor mass and 0.06 Da fragment ion tolerance, static modification of 4plex iTRAQ at N-terminus and lysine, carbamidomethylation at cysteine, and variable modifications of oxidation at methionine. Deamidation at aspargine was applied as a variable modification to identify former glycopeptides. Filters used for global data analysis included peptide rank 1, two peptides per protein, and 2% FDR threshold. Filters used for glycopeptide analysis was peptide rank 1, 1% FDR in Proteome Discoverer. Data was normalized by protein median.

For glycopeptide identification, data was searched in Byonics with the same parameters as mentioned above for proteomic data in the human RefSeq database. Additional parameters for database search were mammalian N-glycans database and offset was set for one and more. Results were filtered using an in-house software a) for presence of two oxonium ions, b) intensity of the highest oxonium ion being greater than maximum reporter ion intensity. C) Results were lastly filtered on the basis of reverse database match, all the peptides were filtered below the score of the highest reverse database match.

### Results

Global Proteomic Analysis.

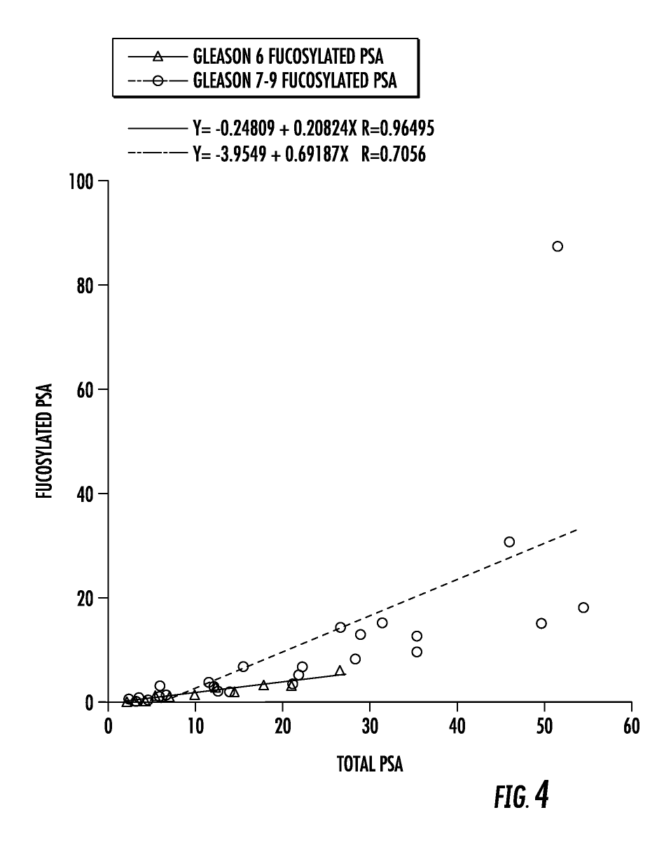

To determine the proteomic profiles of LNCap and PC3 cells, global proteomics was performed using iTRAQ-labelled tryptic peptides from the two cells. Peptides from two dishes of LNCap cells were labeled with 114 and 115 iTRAQ reporter ions to determine the reproducibility of the platform and PC3 cells peptides were labeled with 116 reporter ions (FIG. 7). Labeled peptides were mixed and 400 μg of mixed tryptic peptides were saved for global proteomic analysis. One hundred micrograms of iTRAQ labeled tryptic peptides from LNCap and PC3 were fractionated using bRPLC into 24 fractions. Each fraction was then analyzed by LC-MS/MS to obtain both qualitative and quantitative information on the proteome of LNCap and PC3 cancer cells. At 2% spectral FDR, 8063 protein groups were identified with a minimum of 2 peptides per protein. Two fold differences were considered to be significant changes. Six hundred and thirty seven protein groups were up-regulated in PC3 cells compared to LNCap and 410 proteins were identified to be down-regulated in PC3 cells while no proteins were identified to be up- and down-regulated in the replicated analyses of LNCap cells indicating the false discovery rate of less than 0.01%. Distribution of 8063 global proteins between LNCap replicates and PC3/LNCap cells was represented in the histogram (FIG. 8). In case of LNCap replicate analysis, the distribution proteins were located in the interval between −1 and +1 (on the log 2 scale) (FIG. 8). In case of the PC3/LNCap, distribution of protein ratio beyond −1 and +1 suggested the changes of PC3 cells comparing to LNCap cells.

When comparing the identified global proteins with the genes that are known to involve in glycan synthesis pathways, we found 191 identified proteins were enzymes that are involved in glycan biosynthesis. Fifteen proteins were up-regulated with over 3-fold increase for hyaluronan synthase 3 (HAS3), bifunctional 3′-phosphoadenosine 5′-phosphosulfate synthase 2 (PAPSS2), phosphoglucomutase-1 (PGM1), and alpha-(1,6)-fucosyltransferase (FUT8) in PC3. Another 17 proteins were down-regulated at least 2-fold in PC3 cells comparing to LNCap cells (Table 1). This indicates that there are significant differences in glycan structures between the two cell lines.

Glycoproteomic Analysis.

To determine which of the changed proteins in PC3 cells were glycoproteins, glycosite-containing peptides from the same iTRAQ labeled tryptic peptides from lysed cells for global proteomic analysis were isolated using SPEG. This eliminated variation due to sample preparation between global proteomic and glycoproteomic analyses.

Once glycosite-containing peptides were enriched, peptides were analyzed via LC-MSMS. MS/MS data was searched using SEQUEST against RefSeq and filtered at 1% FDR. Peptides identified were further filtered for deamidation and the presence of the consensus sequence NXS/T to identify glycosite-containing peptides. In this study, we were able to identify 1810 N-linked glycosite-containing peptides from 653 glycoproteins.

Based on the replicate analysis of LNCap cells, a 2-fold threshold was set to determine the number of glycoproteins differentially expressed between the two prostate cancer cell lines. Among the 653 glycoproteins identified and quantified, 97 and 79 glycoproteins contain glycosites that are significantly up- and down-regulated in PC3 as compared to LNCap, while the changes of the replicate analyses of LNCap cells were 1 and 0 glycoproteins up- and down, indicating a false positive rate for significantly changed glycoproteins is 0.1%. A slightly higher number of up-regulated glycoproteins in PC3 cells indicated a moderate increase in glycoprotein expression and/or glycosylation occupancy in PC3 cells. Differentially expressed glycoproteins were compared to glycoproteins without changes using David annotation tool to facilitate biological interpretation in a network context. Down-regulated glycoproteins in PC3 are highly represented in the lysosome pathway, where CTSL2 and LGMN enriched lysosomal acid hydrolases, IDUA, NAGA, FUCA1, HEXA, MANB enriched as glucosidase and AGA, P SAP, GM2A, NRAMP and CLNS are also belong to this pathway and down-regulated. Other glycan degradation pathway is also enriched in PC3 down-regulated glycoproteins including AGA, FUCA1, FUCA2, HEXA, and MANBA that are specifically in N-glycan degradation pathway. Up-regulated glycoproteins in PC3 compared to LNCap are enriched extracellular matrix (ECM) pathway with LAMB1, CD44, CD47, ANPEP, ITGB4, ITGA1, ITGA2, ITGA3, RELN and HSPQ2 glycoproteins up-regulated. Hematopoietic cell lineage, cell adhesion molecule and focal adhesion pathway proteins were also enriched in up-regulated glycoproteins with p-values below 0.005.

Glycoprotein Differences Between LNCap and PC3 and Glycosylation Occupancy.

Although 176 glycoproteins underwent 2 fold changes between PC3 and LNCap cells, it is unclear whether the changes were due to changes in protein expression or in glycosylation site occupancy. To resolve the above dilemma, we compared the glycoprotein changes identified from glycoproteomics to those identified from global proteomic analysis.

The comparison of the ratio of glycoprotein to the global protein between the cell lines informs differential glycosylation occupancy on the glycoproteins. Plotting the histogram of glycoprotein/protein ratio between PC3 and LNCap, the distribution revealed that vast majority of the proteins i.e., 88.1% are located within interval −1 and +1 interval (on the log 2 scale) indicating that these proteins were regulated in protein expression level and glycoproteins likely contained similar glycosylation occupancy between the two cell lines (FIG. 8). However ratio of 11.9% of glycoproteins were located outside the interval of −1 and +1 suggesting significant changes in glycosylation of these proteins. The data showed that among the glycoprotein changes, majority of glycoprotein changes were due to the differential protein expression and there was a subset of glycoprotein changes were due to differential glycosylation occupancy.

Among the 97 up-regulated glycoproteins from PC3 compared to LNCap, 74 glycoproteins were also identified in global proteomics, out of which 55 proteins were up regulated in total protein level for over 2 fold and 4 glycoproteins were identified with decreased total protein expression. Out of 79 glycoproteins identified with a 2 fold decreased expression in PC3 cells, 67 glycoproteins were also quantified in global protein level and 27 proteins showed at least 2 fold decrease and 7 glycoproteins showed at least 2-fold increase in their total protein level in PC3 cells. These results suggested that in additional to the expression of protein, change in glycosylation occupancy, in part, could be the cause of the glycoprotein changes. Change in occupancy was estimated by calculating the ratio of fold change in N-glycosite containing peptides to that in total protein levels between two cell lines. From this analysis, 23 glycoproteins were identified with more than 2 fold change in glycosite-containing peptide ratio comparing to global protein levels (Table 2).

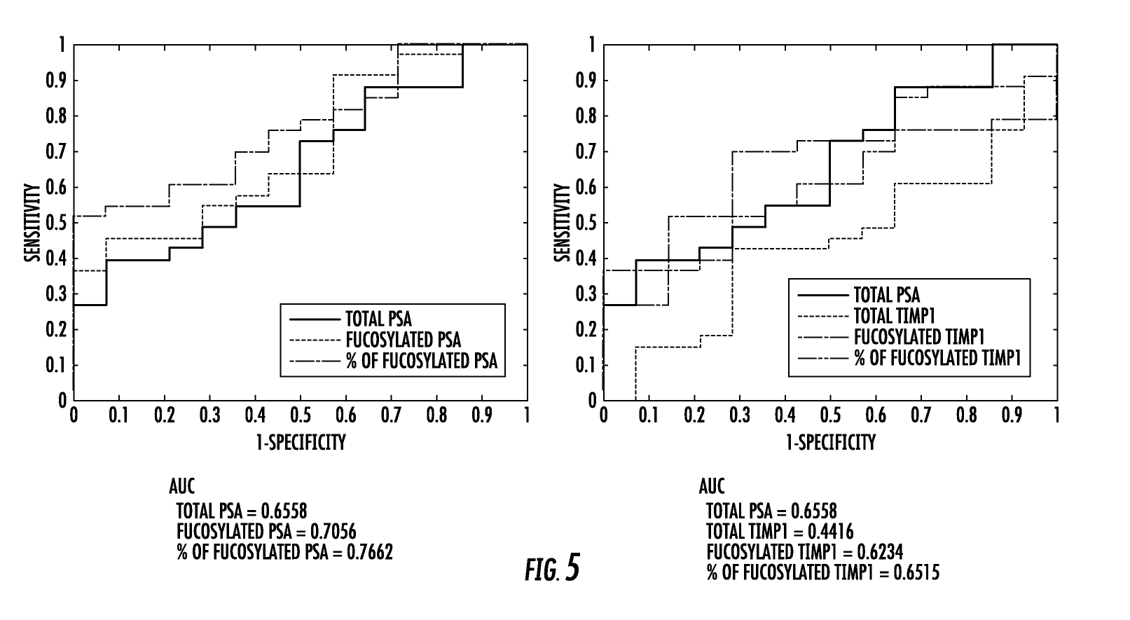

Twenty-three glycoproteins with differential ratios of glycosite compared to total protein levels were due to different glycosylation occupancies of the glycosites and must have partial glycosylation in at least one cell line if not in both. In the case of identified glycoprotein CD63, the observed protein ratio of CD63 is 1.17, however, the observed glycosite ratio is 3.45 quantified with a total of 90 MS/MS spectra. FIG. 9A represents a MS/MS from CD63 peptide identified in global proteomic analysis and FIG. 9B is a MS/MS representing a CD63 glycosite-containing peptide identified using SPEG method. To determine whether the differences in glycoprotein ratio comparing to global protein levels was due to partial glycosylation at the glycosylation site, the global data was searched for the non-glycosylated peptides from global proteomic data that contained sequences of the glycosite-containing peptides identified from glycoproteomics using SPEG method.

Subsequently, we identified 71 spectra for peptides containing the same N-linked glycosylation sites but without glycan attached or Asn to Asp conversion as they were identified by glycoproteomic analysis in global proteomic data. The identification of non-glycosylated peptides existing in both unmodified form and glycosylated forms indicated that these peptides were partially glycosylated and the glycosylation sites were not fully occupied. This may be part of the reasons that different ratios were observed for glycoproteins comparing to those from global proteomics. The extent of glycosylation of a particular site might vary in different cell lines.

Glycopeptide Identification.

To determine the glycosylation heterogeneity at specific glycosylation sites, we further analyzed the global proteomics data to identify the intact glycopeptides. MS/MS spectra from intact glycopeptides have unique signatures of fragmentation in HCD fragmentation technique, in which, oxonium ions are generated along with peptide and peptide+HexNAc fragment ions and were used to identify glycopeptides. In global proteomics data analysis, spectra of glycopeptides had unique signatures. Interestingly in the MSMS spectra of only peptides without glycan, iTRAQ reporter ions at m/z 114, 115, 116 and 117 were generally the base peak with the highest intensities due to HCD fragmentation. For glycopeptides with glycan attached, oxonium ion intensities are observed to be higher than intensities of iTRAQ reporter ions, and diagnostic oxonium ions were the base peak in MSMS. In previous study similar phenomenon was observed when tandem mass tags (TMT) were used for analysis of bovine fetuin and glycosidic bond is preferentially cleaved in HCD over amides and reporter ions. To extract MSMS spectra of glycopeptides from 24 fractions, the important characteristic of oxonium ion intensities being greater than highest reporter ion intensity was used. Another filter was applied in MSMS data of minimum of two out of top five most abundant peaks in MSMS needed to be oxonium ions 138, 163, 204, 274, 292 and 366. This reduced and almost eliminated false positives of a peptide MSMS as a glycopeptide.

With these stringent filters, we were able to identify 3670 MS/MS spectra meeting glycopeptide threshold and assigned to glycopeptides by Byonic in front database and 469 MS/MS were assigned in reverse database. For glycopeptide identification using highest score of matched spectra to reversed database as cut of score, we considered glycopeptides with score greater than score for reverse database match as identified glycopeptides (with <0.05% FDR in spectral level, considering 1 reverse database match), we were able to assign 2022 MSMS spectra to N-glycopeptides to 1145 unique glycopeptides from 227 protein groups. These glycopeptides were made up of 67 glycans.

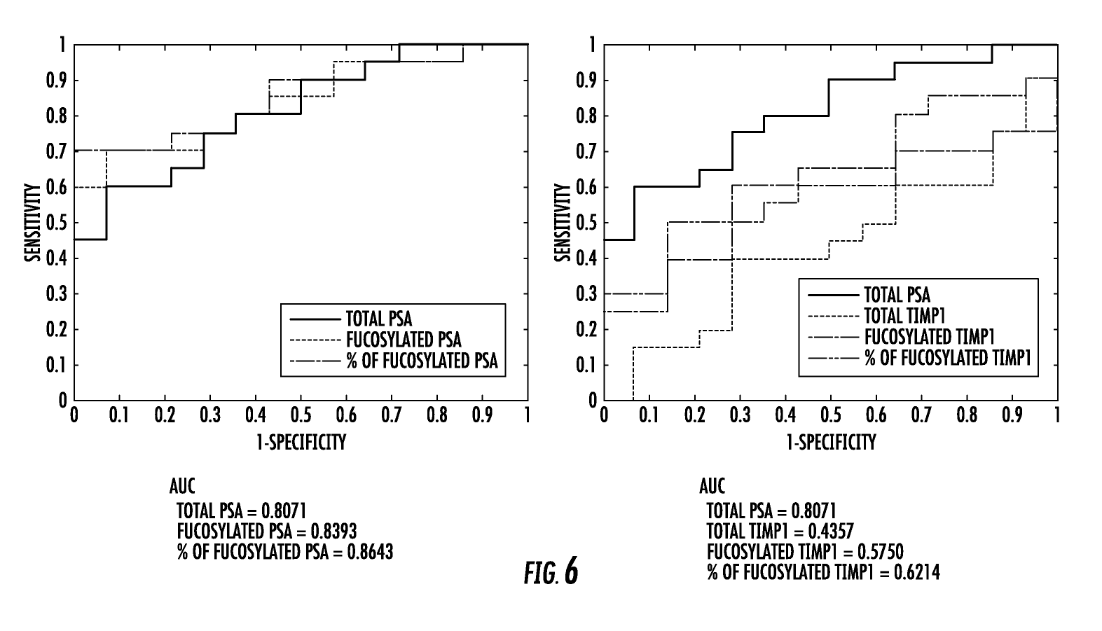

We identified glycopeptides containing 67 different glycan structures including high mannose glycans, fucosylated glycans, sialylated glycans, and sialylated and fucosylated glycans. Most of the MS/MS spectra were assigned to glycosites containing fucosylated or high mannose type N-glycans. We next quantified the glycopeptides containing specific glycans using the iTRAQ reporter ions from the MS/MS results for each glycopeptides. The glycopeptides identified with a minimum of 5 spectra were quantified using iTRAQ report ions to ensure the accuracy. The glycopeptide quantification was then normalized with the same factor determined in global proteomic analysis as the glycopeptide data was extracted from the global proteomic analysis. The glycosites exhibiting both high mannose and fucosylated glycan structures were represented in FIG. 10. The data showed that each glycosite contained unique glycan structure profile with different levels of high mannose and fucosylated glycan structures. The relative abundance of high mannose and fucosylated glycans is independent of protein abundance and glycosylation levels of each glycosites, indicating the glycosylation heterogeneity at each glycosylation site.

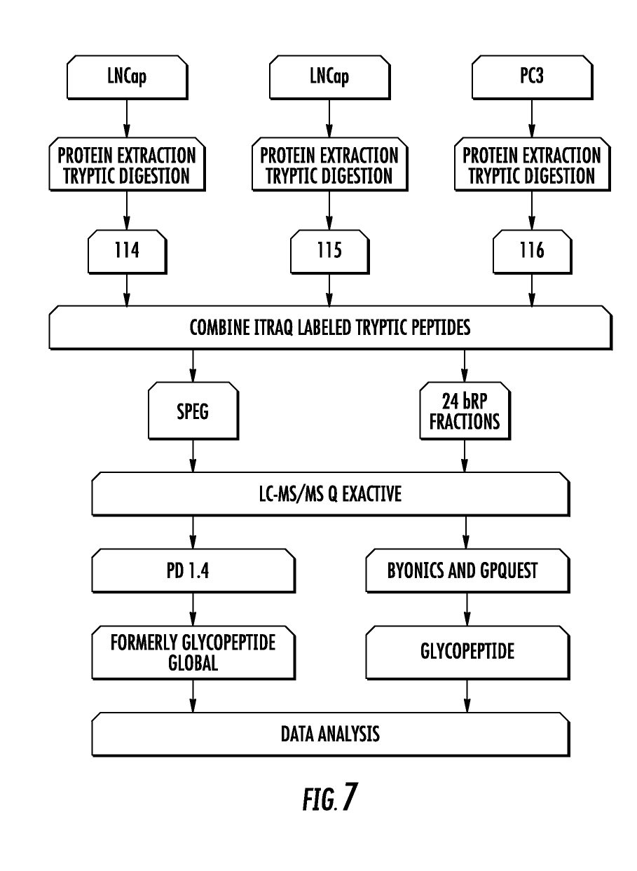

A change in fucosylation site occupancy was observed for several glycosites including protein adipocyte plasma membrane associated protein (APMAP). The relative levels of global protein and glycosylation site, AGPN#GTLFVADAYK (SEQ ID NO:1), of APMAP showed no change between LNCap and PC3 in both protein and glycosite levels identified and quantified by global proteomics and glycosite analysis using SPEG and iTRAQ (FIG. 11A). Using the b and y ions of peptide fragmentation from the glycosite, additionally glycopeptides having the same peptide fragmentation along with high mannose glycans and fucosylated glycans were identified. Glycopeptide with Man₅GlcNAc₂ was identified but the iTRAQ report ion intensity suggested a decrease in occupancy with Man₅GlcNAc₂ at the glycosite in PC3 (FIG. 11B). FIG. 11C was a MSMS of same glycosite containing Fuc₁Man₅GlcNAc₂ glycan, however in this case the intensity of iTRAQ reporter ions suggested increase in occupancy of Fuc₁Man₅GlcNAc₂ glycan at the same glycosite. We identified 8 additional glycopeptides on the same N-linked glycosite from APMAP and observed a various high mannose glycoforms decreased in PC3 compared to LNCap. However fucosylated glycopeptide ratio was up in PC3 cells (Table 4).

TABLE 4
Summary of the Glycopeptide, AGPN#GTLFVADAYK
(SEQ ID NO: 1), with Different Glycoforms.

| | | | | | | | Glycopeptide | |
|---|---|---|---|---|---|---|---|---|
| Acces- | | | | Hex- | Fu- | | LNCap/ | PC3/ |
| sion | Spectra | | | ose | cose | HexNAc | LNCap | LNCap |
| 24308201 | 1 | 3 | 3 | 1 | 0 | 1.46 | 5.44 | 0.99 |
| 24308201 | 2 | 2 | 3 | 1 | 0 | 1.04 | 2.37 | 0.99 |
| 24308201 | 2 | 4 | 3 | 1 | 0 | 1.42 | 7.37 | 0.99 |
| 24308201 | 1 | 2 | 5 | 1 | 0 | 1.14 | 4.33 | 0.99 |
| 24308201 | 2 | 5 | 3 | 1 | 0 | 1.09 | 8.47 | 0.99 |
| 24308201 | 5 | 2 | 5 | 0 | 0 | 1.17 | 0.74 | 0.99 |
| 24308201 | 5 | 2 | 6 | 0 | 0 | 1.09 | 0.39 | 0.99 |
| 24308201 | 2 | 2 | 8 | 0 | 0 | 0.94 | 0.51 | 0.99 |
| 24308201 | 2 | 2 | 9 | 0 | 0 | 1.48 | 0.48 | 0.99 |
| 24308201 | 1 | 2 | 7 | 0 | 0 | 1.12 | 0.94 | 0.99 |

Another protein, prosaposin (PSAP), was identified in SPEG analysis with 6 different glycopeptides from 4 unique glycosylation sites. Out of which five glycopeptides were down-regulated in PC3 cells. Overall protein ratio of PSAP between PC3 and LNCap was 0.85 suggesting no significant change at protein expression level. Upon glycopeptide analysis, the glycosite, NLEKN#STKQEILAALEK (SEQ ID NO:2), had an increased level in fucosylation and the ratio of PC3 versus LNCap was 3.95 with 8 PSM indicating alteration of glycosylation in a site specific manner.

The change in glycoforms could be a result of changes in glycoprotein expression which are glycosylated or protein expression of enzymes that are involved in glycosylation. Quantitative analysis identified 191 proteins that were involved in the, substrate synthesis, glycan branching or elongation, or degradation of N-glycans. One of the glycosylation enzymes identified, alpha-(1,6)-fucosyltransferase (FUT8), is up-regulated at protein level in PC3 by 3.4 fold compared to that in LNCap. Alpha-L-fucosidase (FUCA1) involved in glycan degradation was down-regulated in PC3 cells by more than 2 folds (Table 5). This indicates that change in fucosylation observed in PC3 might be due to changes in expression of fucosyltransferase and alpha-L-fucosidase.

TABLE 5
Changes in Glycosylation Genes form PC3 Cells

| Accession # | Gene Name | Description | Protein Type | PC3/LNCap |
|---|---|---|---|---|
| 20302153 | HAS3 | hyaluronan synthase 3 isoform a | Glycan-transferase | 6.27 |
| 20302155 | HAS3 | hyaluronan synthase 3 isoform b | Glycan-transferase | 6.27 |
| 34447231 | PAP552 | bifunctional 3′-phosphoadenosine 5′-phosphosulfate synthase 2 isoform a | Nuc. Sugar | 3.46 |
| 02912492 | PAP552 | bifunctional 3′-phosphoadenosine 5′-phosphosulfate synthase 2 isoform b | Nuc. Sugar | 3.46 |
| 21361621 | pgm1 | phosphoglucomutase-1 isoform 1 | Nuc. Sugar | 3.45 |
| 30410726 | fut8 | alpha-{1,5}-fucosyltransferase isoform a | Glycan-transferase | 3.41 |
| 4502343 | B3galnt1 | UDP-GalNAcbeta-1,3-N-acetylgalactosaminyltransferase 1 | Glycan-transferase | 2.76 |
| 190014632 | gne | bifunctional UDP-N-acetylglucosamine 2-epimerase/N-acetylmannosamine | Nuc. Sugar | 3.71 |
| | | kinase isoform 1 | | |
| 4885285 | gne | bifunctional UDP-N-acetylglucosamine 2-epimerase/N-acetylmannosamine | Nuc. Sugar | 2.71 |
| | | kinase isoform 2 | | |
| 11321585 | GNB1 | guanine nucleotide-binding protein G(0/G(S)/G(T) subunit beta-1 | Nuc. Sugar | 2.29 |
| 194007330 | Hk3 | hexokinase-3 | Nuc. Sugar | 2.27 |
| 42516563 | UXS1 | UDP-glucoronic add decarboxylane 1 isoform 2 | Nuc. Sugar | 2.13 |
| 240255483 | SULF2 | extracellular sulfatase Sulf-2 isoform a | Glycan Degradation | 2.11 |
| 240255478 | SULF2 | extracellular sulfatase Sulf-2 isoform b | Glycan Degradation | 2.11 |
| 13027378 | gnpda1 | glucosamine-6-phosphate isomerase 1 | Nuc. Sugar | 2.02 |
| 84798622 | MANBA | beta-mannosidase | Glycan Degradation | 0.50 |
| 189011548 | A5AH1 | add ceramidase isoform a preproprotein | Glycan Degradation | 0.49 |
| 189011546 | A5AH1 | add ceramidase isoform b | Glycan Degradation | 0.49 |
| 109148550 | NAAA | N-acylethanolamine-hydrolyzing acid amidase isoform 1 | Glycan Degradation | 0.46 |
| 100148548 | NAAA | N-acylethanolamine-hydrolyzing acid amidase isoform 2 | Glycan Degradation | 0.45 |
| 4505235 | MPI | mannose-6-phosphate isomerase | Nuc. Sugar | 0.45 |
| 189011550 | A5AH1 | acid ceramidase isoform c | Glycan Degradation | 0.45 |
| 4504373 | Hexb | beta-hexosaminidase subunit beta preproprotein | Glycan Degradation | 0.43 |
| 119360348 | fucA1 | tissue alpha-1-fucosidase | Glycan Degradation | 0.42 |
| 285002251 | aga | N(4)-(beta-N-acetylglucosaminyl)-L-asparaginose isoform 1 preproprotein | Glycan Degradation | 0.42 |
| 285002253 | aga | N(4)-(beta-N-acetylglucosaminyl)-L-asparaginose isoform 2 preproprotein | Glycan Degradation | 0.42 |
| 38026892 | alg6 | dolichyl pyrophosphate Man9GlcNAc2alpha-1,3-glucosyltransferase | Glycan-transferase | 0.38 |
| 4507303 | SULT1A2 | sulfotransferase 1A2 | xSulfotransferase | 0.37 |
| 4758092 | cths | di-N-acetylchitobiase | Glycan Degradation | 0.36 |
| 189181666 | hexA | beta-beta-hexosaminidase subunit alpha preproprotein | Glycan Degradation | 0.39 |
| 66346698 | Naglu | alpha-N-acetylglucosaminidase | Glycan Degradation | 0.32 |
| 29550921 | SULT1A3 | sulfotransferase 1A3/1A4 | xSulfotransferase | 0.24 |

TABLE 6
A Summary of All the Glycoproteins Identified with Differential Glycosylation
Occupancy Between PC3 and LNCap Cell Lines

| | | | | | | | | | SPEG/ | |
|---|---|---|---|---|---|---|---|---|---|---|
| | | | SPEG | | | GLOBAL | | | GLOBAL | |
| | | | LNCap/ | PC3/ | | LNCap/ | PC3/ | | LNCap/ | PC3/ |
| Accession | GENE ID | Description | LNCap | LNCap | Spectra | LNCap | LNCap | Spectra | LNCap | LNCap |

PC3 glycosylation up

| | | | | | | | | | | |
|---|---|---|---|---|---|---|---|---|---|---|
| 256985102 | TMED7- | TRAM adaptor with GOLD domain isoform 2 | 1.03 | 6.54 | 11 | 0.96 | 0.92 | 30 | 1.08 | 7.08 |
| | TICAM2 | | | | | | | | | |
| 523567561 | TSPAN6 | Tetraspanin-6 | 0.93 | 3.50 | 15 | 0.89 | 0.84 | 3 | 1.05 | 4.15 |
| 383872538 | CD63 | CD63 antigen | 0.94 | 3.15 | 90 | 0.91 | 1.17 | 3 | 1.03 | 2.69 |
| 148539848 | Dsc3 | Desmocollin-3 isoform Dsc3b | 0.84 | 3.09 | 1 | 0.95 | 1.18 | 7 | 0.88 | 2.63 |
| 530416964 | PVR | Poliovirus receptor | 1.05 | 2.81 | 4 | 1.03 | 1.33 | 7 | 1.02 | 2.12 |
| 530414232 | TMX3 | Protein disulfide-isomerase | 0.98 | 2.65 | 19 | 1.08 | 0.65 | 17 | 0.91 | 4.10 |
| 13273311 | POFUT2 | GDP-fucose protein O-fucosyltransferase 2 | 1.11 | 2.50 | 6 | 1.06 | 1.22 | 15 | 1.05 | 2.12 |
| 530399844 | TMTC3 | Transmembrane and TPR repeat-containing | 1.04 | 2.36 | 7 | 0.99 | 1.05 | 27 | 1.06 | 2.24 |
| | | protein 3 | | | | | | | | |
| 530423393 | UGGT2 | UDP-glucose:glycoprotein glucosyltransferase 2 | 1.01 | 2.04 | 13 | 1.02 | 0.95 | 18 | 0.99 | 2.14 |

LNCap glycosylation up

| | | | | | | | | | | |
|---|---|---|---|---|---|---|---|---|---|---|
| 20127446 | ITGB5 | Integrin beta-5 | 1.03 | 0.45 | 4 | 1.02 | 1.19 | 17 | 1.01 | 0.38 |
| 239582761 | Ero1lb | ERO1-like protein beta | 1.05 | 0.44 | 4 | 0.97 | 0.93 | 6 | 1.08 | 0.47 |
| 237648961 | TXNDC16 | Thioredoxin domain-containing protein 16 | 1.03 | 0.41 | 7 | 1.04 | 0.83 | 10 | 0.99 | 0.49 |
| 592513679 | SLC12A9 | Solute carrier family 12 member 9 | 0.99 | 0.40 | 3 | 0.07 | 0.94 | 3 | 1.15 | 0.43 |
| 4505021 | Lrpap1 | Alpha-2-macroglobulin receptor-associated | 1.13 | 0.34 | 20 | 1.06 | 1.01 | 44 | 1.07 | 0.34 |
| | | protein | | | | | | | | |
| 530429119 | HLAA | HLA class 1 histocompatibility antigen | 0.89 | 0.34 | 4 | 0.96 | 2.63 | 26 | 0.93 | 0.13 |
| 52630342 | HLA-C | HLA class 1 histocompatibility antigen, Cw-1 | 0.96 | 0.33 | 12 | 0.96 | 1.03 | 31 | 1.00 | 0.32 |
| | | alpha chain | | | | | | | | |
| 27436891 | Pofut1 | GDP-fucose protein O-fucosyltransferase 1 | 1.08 | 0.31 | 6 | 1.02 | 1.42 | 23 | 1.06 | 0.22 |
| 45007002 | LAS56 | Ceramide synthase 6 | 1.19 | 0.29 | 6 | 1.03 | 0.75 | 7 | 1.15 | 0.38 |
| 93204867 | GPR158 | Probable G-protein coupled receptor 158 | 1.41 | 0.25 | 4 | 1.31 | 0.60 | 13 | 1.08 | 0.41 |
| 11386147 | PSAP | Proactivator polypeptide | 0.87 | 0.25 | 240 | 1.00 | 0.85 | 159 | 0.86 | 0.29 |
| 530397973 | CADM1 | Cell adhesion molecule 1 | 1.28 | 0.25 | 10 | 1.17 | 0.72 | 4 | 1.10 | 0.34 |
| 62460635 | Tm9sf1 | Transmembrane 9 superfamily member 1 | 0.96 | 0.21 | 5 | 0.99 | 0.99 | 23 | 0.97 | 0.42 |
| 522838254 | EOGT | EGF domain O-linked N-acetylglucosamine | 0.93 | 0.25 | 3 | 1.01 | 1.21 | 8 | 0.92 | 0.21 |
| | | transferase | | | | | | | | |

It is contemplated herein that one or more of the biomarker proteins listed in Table 6 can be measured in methods of treatment of patients. Patients who correlate with having aggressive prostate cancer based on an assay evaluating one or more of the proteins described herein (including one or more of Table 6) can be treated with an appropriate therapeutic regimen. In addition, patients who correlate with having non-aggressive prostate cancer based on an assay evaluating one or more of the proteins described herein (including one or more of Table 6) can be treated with an appropriate therapeutic regimen.

To determine the function of FUT8 overexpression in prostate cancer cells, we analyzed LNCap cells with FUT8 overexpression using the same strategy used for the analysis of LNCap and PC3 cells (FIG. 7). The analysis of LNCap cells in replicates showed consistent quantification. Among the 7217 proteins identified in global proteomics, 39 proteins showed different levels in FUT8 overexpressed LNCap cells comparing to parent LNCap cells with 31 proteins, including the overexpressed FUT8, elevated in FUT8 overexpressed cells and 8 proteins decreased associated with FUT8 overexpression in LNCap cells (Table 7).

TABLE 7
Protein Changes in LNCap Cells with Overexpression of FUT8

| Accession | Description | LNCap/LNCap | LNCap-FUT8/LNCap |
|---|---|---|---|
| 1507821 | UDP-glucoronosyltransferase 2B17 | 0.930 | 6.601 |
| 11321577 | 2′-5′oligoadenylate synthase-like protein | 0.991 | 3.486 |
| 30410726 | alpha-(1,6)-fucosyltransferase | 1.013 | 3.030 |
| 4502101 | annexin A1 | 0.976 | 2.982 |
| 74048422 | G-protein coupled receptor 126 | 0.987 | 2.817 |
| 5453541 | anterior gradient protein 2 homolog | 0.983 | 2.815 |
| 31324543 | calcium/calmodulin-dependent protein idnase II inhibitor 1 | 1.106 | 2.798 |
| 13435364 | desmocollin-2 isoform Dsc2a preproprotein | 0.997 | 2.620 |
| 153082755 | interferon-induced protein with tetratricopeptide repeats 2 | 1.095 | 2.534 |
| 4505211 | matrix metalloproteinase-15 preproprotein | 1.008 | 2.388 |
| 100286381 | interferon-induced protein with tetratricopeptide repeats 1 | 0.973 | 2.371 |
| 1507751 | thymidylate synthase | 0.989 | 2.325 |
| 72534658 | interferon-induced protein with tetratricopeptide repeats 3 | 1.044 | 2.314 |
| 8922549 | histone chaperone ASF18 | 0.967 | 2.231 |
| 4502059 | cyclin-dependent kinases regulatory subunit 2 | 0.914 | 2.193 |
| 530405949 | PREDICTED: nuclealor and spindle-associated protein 1 | 0.900 | 2.180 |
| 4505849 | phospholipase A2, membrane associated | 0.958 | 2.137 |
| 5902146 | ubiquitin-conjugating enzyme E2 C | 0.989 | 2.130 |
| 530383979 | PREDICTED: solute carrier family 22, member 3 | 1.123 | 2.127 |
| 530379552 | PREDICTED: G2/mitotic-specific cyclin-B1 | 0.998 | 2.066 |
| 33354285 | paternally-expressed gene 3 protein | 0.979 | 2.054 |
| 7661906 | PCNA-associated factor | 1.039 | 2.037 |
| 226053185 | paternally-expressed gene 3 protein | 0.979 | 2.035 |
| 344925834 | redox-regulatory protein FAM213A | 1.049 | 2.027 |
| 530379625 | PREDICTED: kinesin family member 20A | 1.000 | 1.973 |
| 164698438 | thymidine kinase, cytosolic | 1.027 | 1.910 |
| 116235448 | protein FAM83D | 1.089 | 1.906 |
| 4504897 | importin subunit alpha-1 | 0.987 | 1.889 |
| 189458817 | transferrin receptor protein 1 | 0.998 | 1.831 |
| 217416398 | hyaluronan mediated mobility receptor | 0.938 | 1.793 |
| 18490991 | lymphokine-activated killer T-cell-originated protein kinase | 1.028 | 1.789 |
| 4502173 | prostate-specific antigen isoform 1 preproprotein | 0.994 | 0.728 |
| 40317620 | transmembrane prostate androgen-induced protein | 0.948 | 0.629 |
| 50363237 | kallikrein-2 isoform 2 preproprotein | 0.978 | 0.628 |
| 29171740 | lipid phosphate phosphohydrolase 3 | 1.021 | 0.495 |
| 148747866 | heparan-sulfate 6-O-sulfotransferase 1 | 1.365 | 0.473 |
| 6382064 | prostatic acid phosphatase isoform PAP | 1.004 | 0.466 |
| 13430854 | nuclear RNA export factor 2 | 0.986 | 0.443 |
| 530122196 | PREDICTED: protein POF1B | 0.949 | 0.410 |

The proteins with decreased levels with FUT8 overexpression are particular interesting since they include known prostate cancer clinical markers such as prostate-specific antigen (PSA) and prostatic acid phosphatase (PAP) and other markers such as kallikrein-2 and other secreted proteins. To determine whether the decreased level of these secreted proteins in FUT8 overexpressed LNCap cells was due to the increased secretion of these proteins, we measured the PSA in FUT8 overexpressed LNCap cells and the parental cells without FUT8 using clinical PSA assays. The average percentage of PSA secreted to cell culture medium comparing to in the LNCap cells without FUT8 expression was 20.21%. With FUT8 overexpression, the average percentage of PSA secreted to cell culture medium comparing to in the LNCap cells increased to 28.52%. This indicates that FUT8 overexpression may induce the secretion of fucosylated proteins from prostate cancer cells.

### Discussion

In current study, 1794 unique N-glycosites from 673 glycoproteins were identified and quantified in human prostate cancer cell lines by hydrazide chemistry based proteomics approach to determine the glycosylation alterations between androgen-dependent and androgen-dependent cells. A significant number of identified glycoproteins were differentially expressed between the two cell lines. Among the 174 identified glycoproteins that were altered by at least 2 fold between two cell lines, 82 proteins were also differentially presented by at least 2 folds in global protein expression level. (33 glycoproteins with 2-fold change in glycoproteomic were not identified in the global proteomic analysis of tryptic peptides). Quantified glycoproteins such as TIMP1, CDH11, CD44, PLOD2, ANPEP and LAMB1 identified with elevated level in our study have already been reported as up-regulated or only expressed in PC3 compared to LNCap. Consistency of differentially expressed proteins or genes between previous reports and our analysis further supported that glycoproteomics could facilitate the discovery of altered glycoproteins. Similarly the quantified glycoproteins NCAM2, FOLH1, ACPP and KLK2 as up-regulated in LNCap cells have previously been reported in genomic studies. NCAM2 is proposed to be a novel gene therapy target for the treatment of prostate cancer. FOLH1 also known as prostate specific membrane antigen (PSMA) plays an important role in prostate carcinogenesis and progression. PSMA has been known to be up-regulated upon androgen deprivation. Differentially expressed proteins identified by our approach have potential to be used as candidates like PSMA or NCAM2 for diagnosis or be targets for drug development. PSA or KLK3 was observed in proteomic analysis; however PSA was not identified by SPEG experiment as expected. The N-glycosite-containing peptide of PSA has a length of 2 amino acids after tryptic digest and it is below the minimum molecular weight detectable in our mass spectrometry analysis (700 Da). Another important protein in androgen pathway identified in global analysis and not in SPEG was the androgen receptor, which was identified to be up-regulated in LNCap compared to PC3 in global analysis.

David Annotation tool showed that in expressed glycoproteins “Other N-glycan degradation” pathway was enriched by up-regulation in LNCap. This pathway was enriched by 5 enzymes that were expressed differentially. These five enzymes are responsible for removal of glycans, impacting glycosylation occupancy or changing the glycan structures of glycoproteins in the cells. Among the 5 enzymes, 2 enzymes are fucosidase which are involved in removal of fucose from glycoproteins and glycolipids. These fucosidases were down-regulated in PC3, along with up-regulation of Fut8 and FutII observed in global analysis explaining the change in fucosylation observed in PC3 cells. In prostate tissues, three fucosyltransferases (FUT3, FUT6, and FUT7) have been shown to be elevated in gene expression and overexpression of these fucosyltransferases in PC3 cells showed they may serve as master regulator of prostate cancer cell trafficking and explain the aggressive nature of PC3 cell lines. In our global proteomic study, only two fucosyltransferases (FUT8 and FUT11) were detected. In case of LNCap and PC3 there seems to be due to change in transcription of enzymes of the fucosylation machinery from our RNA microarray analysis (data not shown).

Altered enzymes involved in fucosylation resulted in changes in protein fucosylation in a site specific manner. In the case of PSAP glycosite, NLEKN#STKQEILAALEK (SEQ ID NO:2), both fucosylation and N-glycosylation increased. However in other PSAP glycosite, we did not observe increase in fucosylation or glycosylation. PSAP has been shown to be elevated in patient prostate cancer tissues with advance prostate cancer. Change of PSAP expression along with change in specific glycosylation occupancy may be a good indicator of prostate cancer and needs to be further investigated. PSAP has a signal peptide indicating that it can be secreted. Altered fucosylation on a secreted protein can be used as biomarker similar to alpha-Fetoprotein (AFP). Altered AFP and fucosylated AFP have been shown to differentiate various pathologies of liver fibrosis, cirrhosis and hepatocellular carcinoma (HCC). In 2006 the FDA approved AFP-L3, a fucosylated form of AFP for the early detection of primary HCC. As in the case of PC3 and LNCap, the expression of FUT8 is low in normal liver but increases in HCC. Secreted fucosylated proteins from prostate cancer tissues need to be studied, which can lead to identification of novel biomarkers. Altered fucosylated glycans have been reported in serum glycome from 10 normal patients and 24 prostate cancer patients. However, the origin of the glycan changes is not clear in the previous reports. Gp96 is another glycoprotein exhibited an increase in fucosylation and Gp96 purified from tumors initiates tumor specific ytotoxic T Lymphycytes cells response. The amino acid sequence from tumor and normal tissues are the same, however fucosylation differences between cancerous and normal tissue have been observed, suggesting the role of glycosylation on the cancer specific immune system response and importance of studying the glycosylation. LAMP2 is a unique case that exhibits decrease in fucosylation and increase in sialylation in our study. LAMP2 is a highly glycosylated protein with 16 potential N-linked glycosylation sites. Altered glycosylation may impact protein structure resulting into functional variations and highlights the importance of glycosylation related to disease understanding for diagnosis, prognosis and therapy.

As expected most of the glycoprotein changes observed in specific glycosites were accompanied by changes in protein expression. However few proteins underwent changes in glycosylation without the changes in protein level between LNCap and PC3. In glycoproteomic analysis, N-glycosite-containing peptide from CD63 was more in PC3 than compared to LNCap, and global expression of the protein remained unchanged between two cell lines suggestive of an overall increase in occupancy of CD63 glycosylation in PC3 cells. CD63 N-linked glycosylation has been shown to be important for its interaction with CXCR4 and regulation of CXCR4 trafficking. CXCR4 is a chemokine receptor type 4, CXCR4 expression has been related to metastasis of cancer cells. CD63 altered glycosylation may play a significant role in prostate cancer metastasis and needs further investigation. Other interesting glycoproteins observed was tetraspaninn and integrin beta 5. Tetraspanin is cell surface molecule and play a role in tumor cell motility and angiogenesis and is considered potential therapeutic target. Aberrant glycosylation is associated with pathological conditions including cancer. Tetraspanin was highly glycosylated in PC3 whereas Integrin beta 5 was glycosylated more in LNCap. Tetraspanins are known to interact with integrin, for example CD151 modulates the glycosylation of intergrin alpha5B1 which in turn regulates integrin mediated cell adhesion. The difference in relative abundance of glycosite-containing peptides and the total protein levels indicates that glycosylation occupancy may play an important role on the metastasis potential of cancer cells and the observed differences between the glycosylation occupancy of these two cell lines might explain the difference between their aggressiveness. Integrin beta 5 deficiency results in lower migration and proliferative properties in tumor cells. N-glycosylation role needs to be investigated for integrin beta 5.

Rapid improvements in instrumentation of LC-MS/MS enable high-throughput proteomics to identify thousands of proteins. In current study, increase in sensitivity and speed of LC-MS/MS system has made identification and quantitation of intact glycopeptide from global proteomic analysis possible. However identifying all the possible glycoforms attached to each glycopeptide is still a challenge. Reporter ion intensity from glycopeptides was lower than the unmodified peptide intensity, resulting in decrease in confidence of quantitation. However multiple spectra can convey more reliable information to quantify the glycopeptide as in the case of proteins. In current study, the fragment ions from glycosite-containing peptides were used to validate the glycopeptides from the same glycosite, oxonium ions were used to confirm the glycosylation. In MSMS spectra, limited information was available about the localization and the glycoforms. Based on accurate precursor mass and the glycosite-containing peptide identified, the glycoepeptides with different glycoforms can be extrapolated. However there was limited structures and there is no information about the glycan structure or the linkages between the monosaccharide's forming the glycan.

A thorough investigation of glycan structures on the glycoproteins is required to understand the progression of cancer relation with change in glycosylation. In mass spectrometry, further technological advancements in high throughput LC-MS/MS systems and data analysis is needed, however progress is continuously made in this arena. Quantitative assays need to be developed to validate identified glycosylation by current technologies. Site specific glycosylation changes need to be researched to gain better understandings of the roles of different glycoforms in biological functions. Multiple integrated proteomic strategies are ideal approaches for similar future studies to provide a holistic view to understand the biological changes within a system and could readily be translated to tumor tissue and other cancer systems.

### CONCLUSION

In this study we report, a glycoproteomics-wide comparison of LNCap and PC3 cells, the two major cell models for androgen-dependent and androgen-independent prostate cancer cells with different metastatic potential. The two cell lines were vastly different with many glycoproteins and proteins being expressed. We not only identified differential expression of glycoprotein but also identified changes in glycosylation occupancy in glycoproteins. This is also the first high throughput study of glycan structure determination on different proteins present in LNCaP and PC3 cell lines. We have demonstrated that integratedomics approach for quantification of N-linked glycopeptides in combination with iTRAQ labeling could be useful in determination of altered glycosylation. Identified altered glycosylation can be pursued as a target for biomarkers for early detection of prostate cancer and also be used as therapy targets for cancer. Using the strategy we have started a process for comprehensive glycoprotein analysis of prostate cancer tumor samples, this glycoprotein analysis provides a thorough inside into the post translational modification of N-glycosylation in LNCap and PC3 for further cancer research.

We claim:

1. A method comprising the steps of:
(a) isolating glycoproteins from a serum sample obtained from the patient using a lectin affinity capture assay; and
(b) quantitating the amount of fucosylated PSA from the isolated glycoproteins of step (a) using an immunoassay.

2. The method of claim 1, further comprising the step of generating a report showing the quantitated amount of fucosylated PSA.

3. The method of claim 2, wherein the report further comprises a display of control levels of fucosylated PSA that correlate to aggressive prostate cancer and non-aggressive prostate cancer.

4. The method of claim 1, further comprising the step of identifying the patient as having aggressive prostate cancer based on a statistically significant increase in fucosylated PSA present in the patient sample relative to reference levels that correlate to non-aggressive prostate cancer.

5. The method of claim 1, further comprising the step of recommending, prescribing or treating the patient with, an appropriate therapeutic regimen for aggressive prostate cancer if the quantitated amount of fucosylated PSA correlates to aggressive prostate cancer or recommending, prescribing or treating the patient with, an appropriate therapeutic regimen for non-aggressive prostate cancer if the quantitated amount of fucosylated PSA correlates to non-aggressive prostate cancer

6. A kit comprising:
(a) monoclonal antibodies that each specifically bind a biomarker protein of interest;
(b) biotinylated immunoglobulin G detection antibodies;
(c) biotinylated lectins that specifically bind glycosylated forms of the biomarker protein of interest; and
(d) streptavidin labeled fluorescent markers.

7. The kit of claim 6, further comprising (e) magnetic beads for conjugating to monoclonal antibodies that each specifically bind a biomarker protein of interest.

8. The kit of claim 6, wherein the biomarker protein of interest comprises PSA and the glycosylated form of PSA comprises fucosylated PSA.

9. The kit of claim 8, wherein the biomarker protein of interest further comprises one or more of transmembrane prostate androgen-induced protein, kallikrein-2, lipid phosphate phosphohydrolase 3, heparan-sulfate 6-O-sulfotransferase, prostatic acid phosphatase (PAP), nuclear RNA export factor 2, and protein POF1B.

10. The kit of claim 9, wherein the glycosylated form of the one or more biomarker proteins of interest is fucosylated.
